(19) United States
(12) Patent Application Publication
(10) Pub. No.: US 2017/0119851 A1
(43) Pub. Date: May 4, 2017

(54) METHODS OF TREATING ANOREXIA

(71) Applicants: Juli Erin JONES DAVENPORT, Framingham, MA (US); John Richard Neville HADCOCK, Dedham, MA (US)

(72) Inventors: Juli Erin JONES DAVENPORT, Framingham, MA (US); John Richard Neville HADCOCK, Dedham, MA (US)

(73) Assignee: NOVARTIS AG, Basel (CH)

(21) Appl. No.: 15/312,437

(22) PCT Filed: May 19, 2015
(86) PCT No.: PCT/US2015/031601

§ 371 Date: Nov. 18, 2016

Related U.S. Application Data
(60) Provisional application No. 62/000,480, filed on May 19, 2014.

(51) Int. Cl. A61K 38/22; A61K 9/00

(52) U.S. Cl. CPC A61K 38/22; A61K 9/0043

(57) ABSTRACT

The invention relates to the stimulation of appetite and treatment of such appetite-suppressed conditions as cachexia, and in particular to an intranasal route of administration for appetite-stimulating agents such as peptide compounds and fragments related to or comprising the Agouti-Related Peptide (AgRP).

### FIELD OF THE INVENTION

The invention relates to the stimulation of appetite and treatment of such appetite-suppressed conditions as cachexia, and in particular to an intranasal route of administration for appetite-stimulating agents such as peptide compounds and fragments related to or comprising the Agouti-Related Peptide (AgRP).

### BACKGROUND OF THE INVENTION

Decreased appetite and weight loss are associated with adverse outcomes in multiple conditions, including anorexia nervosa, human aging, cancer, heart failure, chronic obstructive pulmonary disorder and renal failure. Anorexia is often associated with cachexia: a complex metabolic syndrome characterized by excessive loss of muscle mass with or without loss of fat mass that is more than expected for the decreased energy intake. This debilitating condition dramatically shortens lifespan and reduces quality of life.

Under normal circumstances, animals and humans respond to malnourishment with a complex neuroendocrine response that ultimately leads to an increase in appetite, a relative sparing of lean body mass and burning of fat stores, and an overall decrease in basal metabolic rate (Webber & Macdonald, 1994, Brit. J. Nutr. 71:437-447; Ahima et al., 1996, Nature 382:250-252). In contrast, in some diseases a devastating pathological state of malnutrition known as cachexia arises, brought about by a synergistic combination of a dramatic decrease in appetite and an increase in metabolic rate and metabolism of both fat and lean body mass, producing a relative wasting of lean body mass (Tisdale, 1997, J. Natl. Cancer Inst. 89: 1763-1773; Inui, 1999, Cancer Res. 59: 4493-4501; Fong et al., 1989, Amer. J. Phys. 256: R659-R665; Bruera, 1997, Brit. Med. J. 315: 1219-1222; Emery, 1999, Nutrition 15: 600-603). This combination is found in a number of disorders including cancer, cystic fibrosis, AIDS, rheumatoid arthritis, ALS, renal failure, wasting disorders, and hip fracture (Tisdale, 1997, ibid.).

The severity of cachexia in many illnesses may be the primary determining factor in both quality of life, and in eventual mortality (Tisdale, 1997, ibid.; Larkin, 1998, Lancet 351: 1336). Indeed, body mass retention in AIDS patients has a stronger correlation with survival than any other current measure of the disease (Kotler et al., 1989, Amer. J. Clin. Nutr. 50: 444-447). Many different tumor types have been studied and it is a common finding that tumor-bearing animals die from cachexia and exhaustion of metabolic fuels, rather than from metastasis or infection (Svaninger et al., 1987, J. Natl. Cancer Inst. 78: 943-950; Emery, 1999, Nutrition 15: 600-603; Svaninger et al., 1989, Eur. J. Cancer Clin. Oncol. 25: 1295-1302; Emery et al., 1984, Cancer Res. 44: 2779-2784). Cachexia is commonly observed in patients with cancer, particularly in children and elderly individuals (Bruera, 1997, ibid.). The resulting malnutrition and loss of lean body mass reduces the quality of life for the affected individual and compromises recovery by decreasing tolerance to therapy and increasing post-surgical complications (Larkin, 1998, ibid.; Inui, 1999, ibid.).

A key example of anorexia occurs in human aging. Strikingly, elderly frail patients show an almost complete absence of hunger during fasting and postprandial periods (Serra-Prat 2013). Elderly anorexic patients have a three-fold increased risk of mortality, after adjusting for other possible causes of death (Morley et al., 1997, Am J Clin Nutr. October; 66(4):760-73). Up to 1.6 million nursing home residents in the USA have anorexia and >20% require intervention (Morley et al., 2006 Am J Clin Nutr. April; 83(4):735-43). Weight gain in the frail elderly reduces falls and decreases morbidity and mortality following hip fracture and improves overall quality of life (Morley J. E., Am J Clin Nutr. 66:760-763 (1997)).

Tumor growth is associated with profound metabolic and neurochemical alterations, which can lead to the onset of the anorexia cachexia syndrome. Anorexia is defined as the loss of the desire to eat, while cachexia results from progressive wasting of skeletal muscle mass and to a lesser extent adipose tissue, occurring even before weight loss becomes apparent. Cancer anorexia-cachexia syndrome is highly prevalent among cancer patients, has a large impact on morbidity and mortality, and impinges on patient quality of life. However, its clinical relevance is frequently overlooked, and treatments are usually only attempted during advanced stages of the disease (Laviano A. et al, Nat. Clin. Pract. Oncol. 3:158-65 (2005)).

At present there is no pharmacological agent suitable for treating anorexia in the elderly, and current treatment is largely limited to nutritional intervention. Appetite stimulants would greatly benefit the elderly anorexic patient by promoting weight regain. To treat cachexia, it is believed that stimulating appetite alone is not enough, and metabolic changes are also required. Treatment of anorexia and cachexia (associated with aging, cancer, heart failure, COPD, or renal failure) by promoting weight gain, muscle gain, improved physical function, activities of daily living, and quality of life would offer significant clinical benefits to many patients.

Attempts at drug therapy for cachexia with a variety of agents have met with limited success (DeConno et al., 1998, Eur. J. Cancer 34: 1705-1709; Windisch et al., 1998, Ann. Pharmacother. 32: 437-445; Rivandeneria et al., 1999, Nutr. Cancer 35: 202-206; McCarthy, 1999, Res. Nurs. Health 22: 380-387). The most widely utilized agent, megestrol acetate (a progesterone analog), has shown some promise in reversing weight loss, but this is primarily due to increases in fat mass and water retention, rather than preservation of lean body mass (Strang, 1997, Anticancer Res. 17: 657-662). Another approved anorexia therapy, dronabinol (a cannabinoid analog), is approved in AIDS-related wasting but provides limited efficacy and has multiple side effects.

The Blood-Brain Barrier (BBB) can allow small (about less than 500 Da), lipophilic molecules from the bloodstream to enter the CNS (Pardridge, Arch Neurol. 2002; 59:35-40), but restricts the passage of large molecules (Pardride, NeuroRx. 2005 January; 2(1): 1-2. 2005). Many large-molecule drugs or genes are prevented thereby from reaching the brain and treating CNS disorders such as Parkinson's disease, Alzheimer's disease, depression, stroke, and epilepsy (Pardridge, NeuroRx. 2005 January; 2(1): 3-14).

Devices for delivering a compound to the olfactory region of the nasal cavity, i.e., for administering a compound intranasally, now exist and have demonstrated an ability to achieve direct nose-to-brain delivery. These devices facilitate the crossing of the BBB by therapeutic agents, e.g., for treating such disorders as cachexia (e.g., CNS disorders such as anorexia-cachexia), and can achieve surprising and unexpected levels of efficacy, even when administering known therapeutic agents.

The Agouti-Related Peptide (AgRP) plays a central role in energy balance by reducing signaling through the hypothalamic melanocortin receptors (MCRs) 3 and 4, in turn stimulating feeding and decreasing energy expenditure.

The Agouti-Related Peptide (AgRP) is a signaling molecule made up of 132 amino acids that is post-translationally processed into its active or mature form of 50 amino acids with 5 disulfide bridges (also indicated as AgRP 83-132). Mature AgRP, produced by endoproteolytic processing, contains a central region that folds as an inhibitor cystine knot (ICK) stabilized by a network of disulfide bonds; this domain alone carries the molecular features for high affinity melanocortin receptors (MCR) binding and inverse agonism.

The novel discovery that the intranasal (IN) administration of AgRP increases appetite and food intake in lean rats and mice has led to the methods of the present invention. As compared to other appetite stimulants, AgRP therapy, e.g., via IN administration, may be useful for the treatment of anorexia cachexia.

### SUMMARY OF THE INVENTION

The invention relates to the present discovery of the appetite stimulating properties of intranasally (IN) administered AgRP, which has among other things, been shown to increase appetite and food intake in lean rodents.

The active form of AgRP is a 50 amino acid peptide with 5 disulfide bridges (also indicated as AgRP 83-132). It is an inverse agonist of the melanocortin receptors MC3R and MC4R. AgRP also blocks the interaction of the endogenous melanocortin receptor agonist, melanocyte stimulating hormone, MC3R and MC4R. AgRP is a known appetite stimulant when delivered to the brain through intracerebroventricular direct central administration. In addition, due to its molecular size, structure and physical properties systemic administration of AgRP cannot readily bypass the Blood-Brain Barrier (BBB) in order to stimulate appetite.

The present invention is drawn to methods of treating a patient exhibiting one or more wasting disorders such as anorexia cachexia, anorexia of the aged, anorexia nervosa, cachexia associated with cancer, cachexia associated with AIDS, cachexia associated with heart failure, cachexia associated with cystic fibrosis, cachexia associated with rheumatoid arthritis, cachexia associated with kidney disease, cachexia associated with COPD, cachexia associated with ALS, cachexia associated with renal failure or cachexia associated, or hip fracture, and in reducing the mortality and morbidity of critically ill patients, comprising administering to said patient in need of such treatment a therapeutically effective and intranasally delivered amount of one or more human AgRP polypeptides, analogs, variants, or peptides or pharmaceutical compositions thereof.

The present invention is also drawn to methods of treating a patient exhibiting one or more of the following: Cushing's syndrome, Hypercortisolism, the ectopic ACTH syndrome, the change in adrenocortical mass, primary pigmented nodular adrenocortical disease (PPNAD) Carney complex (CNC), the cortisol-induced mineralocorticoid excess, conditions associated with post-traumatic stress disorder, hirsutism, thin skin, myopathy, osteoporosis, increased tissue fragility, poor wound healing, hypertension, diabetes mellitus, low serum potassium, and low eosinophils and lymphopenia.

Similarly, as used herein, the term “compositions of the present invention,” as well as any like terms, when applying to AgRP, define proteins, peptides, polypeptides, analogs, variants, and pharmaceutical compositions thereof embodied by the present methods and compositions.

These and other aspects of the invention will be elucidated in the following detailed description of the invention.

### BRIEF DESCRIPTION OF THE DRAWINGS

FIGS. 1A-B are graphical representations of an unpaired t-test comparing IN administered saline and 57 μg AgRP, at 4 h (FIG. 1A: t=2.2, p<0.05) and 24 h (FIG. 1B: t=2.7, p<0.05) after dosing in lean mice. Data graphed as mean±SEM.

FIGS. 2A-B are graphical representations of an unpaired t-test comparing subcutaneous (SC) administered PBS and 57 μg AgRP, at 4 h (FIG. 2A: t=0.7, p>0.5) and 24 h (FIG. 2B: t=0.4, p>0.5) after dosing in lean mice. Data graphed as mean±SEM.

FIG. 3 is a graphical representation showing an plasma exposure of AgRP in mice following intranasal (IN) or subcutaneous (SC) administration of 57 μg AgRP.

FIGS. 4A-B are graphical representations of an unpaired t-test comparing saline and 57 μg AgRP resulted in a significant effect at 4 h (FIG. 3A: t=2.6, p<0.05) and both 57 μg and 171 μg AgRP elicited a significant increase in food intake at 24 h (FIG. 3B: t=2.1, and t=1.8 p<0.05). Data graphed as mean±SEM.

FIG. 5 is a graphical representation showing an increase in AgRP exposure in the rat hypothalamus following intranasal (IN) administration of 57 μg AgRP compared to saline treated lean rats.

### DETAILED DESCRIPTION OF THE INVENTION

The invention relates to the present discovery of the appetite stimulating properties of intranasally (IN) administered AgRP, which has, inter alia, been shown to increase appetite and food intake in lean rats and mice.

The Agouti-Related Peptide (AgRP) is a signaling molecule made up of 132 amino acids that is post-translationally processed into its active or mature form of 50 amino acids with 5 disulfide bridges (also indicated as AgRP 83-132). Mature AgRP, produced by endoproteolytic processing, contains a central region that folds as an inhibitor cystine knot (ICK) stabilized by a network of disulfide bonds; this domain alone carries the molecular features for high affinity melanocortin receptors (MCR) binding and inverse agonism.

AgRP is an inverse agonist of the melanocortin receptors MC3R and MC4R (Seeley R J, et al. (2004) Annu Rev Nutr. 24:133-49) (Reizes O, et al. (2003) Ann N Y Acad Sci. June; 994:66-73). AgRP and the melanocortin receptors are highly conserved across mammals. There are no reported differences in the biological activity of AgRP across species.

AgRP plays a central role in energy balance by reducing signaling through the hypothalamic melanocortin receptors (MCRs) 3 and 4, in turn stimulating feeding and decreasing energy expenditure. Deletion of MC3R and/or MC4R in mice all lead to increased fat and/or muscle mass suggesting that simultaneous blockade of both targets would lead to the greatest benefit for the anorexic patient with low body weight. Indeed, mice that have deletions of both MC4R and MC3R exhibit greater obesity than single deletions of either receptor (Raffin-Sanson et al., 2001 Eur J Endocrinol. March; 144(3):207-8).

Furthermore, MC3R and MC4R facilitate weight gain by different physiological mechanisms. While both impact appetite, signaling through MC4R alters energy expenditure while MC3R signaling shifts substrate utilization in favor of fat oxidation. In preclinical models, caloric restriction increases AgRP gene expression in the arcuate nucleus of the hypothalamus by as much as 5-fold (Rijke, et. al. 2005 J Mol Endocrinol Oct. 1, 2005 35 381-390). AgRP is a known appetite stimulant when delivered to the brain through intracerebroventricular (ICV) administration. AgRP requires direct delivery to the central nervous system for efficacy, as peripheral administration of AgRP is ineffective at stimulating appetite (Seeley R J, et al. (2004) Annu Rev Nutr. 24:133-49). Centrally delivered AgRP by ICV administration is a potent appetite stimulant in rodents, sheep and primates (Hagan M M, et al (2000) Am J Physiol 279:R47-R52) (Koegler et al., (2001) Endocrinology. June; 142(6):2586-92) (Wagner C G, et al. (2004) Neuroendocrinology. 80(4):210-8), indicating that there is conservation of this pathway across species. In rodents, a single ICV administration of AgRP results in increases in food intake for several days.

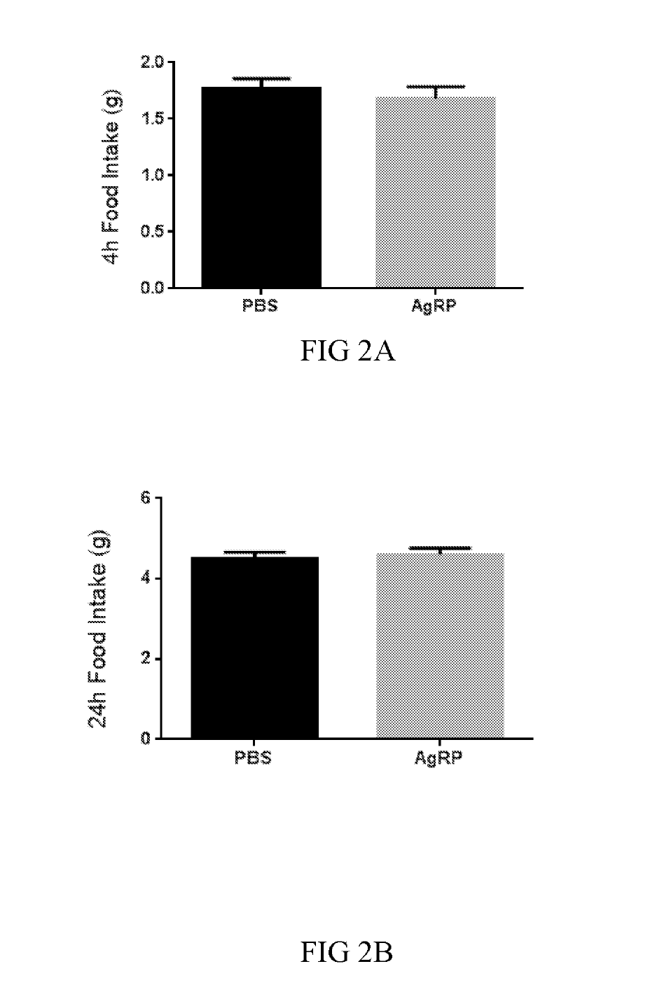

From animal models it is known that AgRP levels decline as rodents age, yet they maintain the orexigenic responses to AgRP administration (Wolden-Hanson et al. 2004 Am J Physiol Regul Integr Comp Physiol 287:R138-R146). Neurons of the arcuate nucleus of the hypothalamus produce AgRP and project to deep brain structures known to modulate feeding and energy balance. Since peripherally-administered AgRP does not stimulate food intake, the peptide needs to be delivered centrally to increase food intake (FIGS. 2A and 2B).

Due to its molecular size, structure and physical properties, however, AgRP, when systemic administered through conventional delivery methods, cannot readily bypass the blood brain barrier to stimulate appetite. The methods of the present invention represent treatment of wasting disorders such as cachexia (e.g., anorexia cachexia), by intranasally administering AgRP. The targets of AgRP are located behind the blood brain barrier (BBB) in deep hypothalamic structures such as the paraventricular nucleus of the hypothalamus (PVH). Therefore, AgRP, when delivered intranasally to access the brain, in particular the hypothalamus, where it exerts its orexigenic effect, can restore normal hunger and promote weight regain in the anorexic patient. IN administration provides a non-invasive method of bypassing the blood brain barrier to deliver peptides, such as AgRP, to the brain.

ICV injection of AgRP increases food intake in rodents, sheep, and monkeys (Hagan M M, et al (2000) Am J Physiol 279:R47-R52) (Koegler et al., (2001) Endocrinology. June; 142(6):2586-92) (Wagner C G, et al. (2004) Neuroendocrinology. 80(4):210-8). ICV AgRP can decrease energy expenditure, and increase total body mass, fat mass and lean body mass (i.e., muscle) in heart failure cachexia (Scarlett J M, et al. (2010) Journal of Endo. 206, 121-130), chronic kidney disease (Cheung W W, et al. (2012) Am J Physiol Renal Physiol. 303(9):F1315-24), cancer cachexia and anorexia nervosa mouse models (Hillebrand J J, et al. (2006) Eur Neuropsychopharmacol; 16(6):403-12.). Furthermore, very old animals retain sensitivity to the orexigenic effects of AgRP similar to young animals (Wolden-Hanson T, et al. (2004) Am J Physiol Regul Integr Comp Physiol.; 287(1):R138-46).

These pathways are also relevant for humans. In the human brain, fiber-like staining of AgRP is adjacent to MC4R-positive cells in the PVH (Alkemade et al., 2012 J Clin Endocrinol Metab. June; 97(6):E925-33) (Siljee et al. 2013 Eur J Endocrinol. February 15; 168(3):361-9). Mutations in MCR4 are the most common genetic cause of obesity (˜1% of BMI>BMI 30) (Srinivasan S, et al. (2004) J Clin Invest; 114(8):1158-64). In humans, peripheral AgRP levels are elevated after fasting and suppressed after a meal (Shen C P, et al. (2002) J Neuroendocrinol; 4(8):607-10). In addition, cerebrospinal fluid (CSF) AgRP levels are elevated in pregnant compared to non-pregnant women, consistent with the increased food intake necessary to achieve the positive energy balance that is essential during pregnancy (Page-Wilson G, et al. (2013) J Clin Endocrinol Metab; 98(1):264-71).

Among the known collection of natural and synthetic orexigenic peptides, AgRP exhibits the greatest overall duration of effect. For example, a single ICV dose (1.0 nM) of neuropeptide Y (NPY) rapidly stimulates feeding beyond that of an equivalent dose of AgRP, but its effects quickly dissipate and feeding returns to baseline after 24 hours (Flynn et al., 1999; Hagan et al., 2000).

Because of its unique behavior, AgRP is considered to be an important lead in the development of drugs for treating cachexia (Marks et al., 2001). Cachexia is a state of negative energy balance that often arises with cancer, AIDS, kidney failure and leads to malnutrition and loss of body mass (Grossberg et al., 2010; Krasnow and Marks, 2010). Maintaining positive energy balance, on the other hand, correlates strongly with the outcome of cancer patients undergoing radiation or chemotherapy. Consistent with the role of the melanocortin system in maintaining energy balance, animal models driven to cachexia by tumors or administration of lipopolysaccharide (LPS) resume normal feeding and body weight from the administration of MC4R antagonists, including AgRP.

The present invention is drawn to methods of treating a patient exhibiting one or more wasting disorders such as anorexia cachexia, anorexia of the aged, anorexia nervosa, cachexia associated with cancer, cachexia associated with AIDS, cachexia associated with heart failure, cachexia associated with cystic fibrosis, cachexia associated with rheumatoid arthritis, cachexia associated with kidney disease, cachexia associated with COPD, cachexia associated with ALS, cachexia associated with renal failure or cachexia associated, or hip fracture, and in reducing the mortality and morbidity of critically ill patients, comprising administering to said patient in need of such treatment a therapeutically effective and intranasally delivered amount of one or more human AgRP polypeptides, analogs, variants, or peptides or pharmaceutical compositions thereof.

### DEFINITIONS

Various definitions are used throughout this document. Most words have the meaning that would be attributed to those words by one skilled in the art. Words specifically defined either below or elsewhere in this document have the meaning provided in the context of the present invention as a whole and as are typically understood by those skilled in the art.

As used herein, the term “AgRP,” and like terms refer to the Agouti-Related Peptide, i.e., a signaling molecule made up of 132 amino acids that is post-translationally processed into its active or mature form of 50 amino acids with 10 cysteine residues and 5 disulfide bridges, AgRP (83-132), which plays a role as an inverse agonist of the melanocortin receptors MC3R and MC4R. Said term in all instances includes salts thereof. In some embodiments, AgRP can be in an amide form, e.g., formed by joining the —CO2H end of one amino acid with the —NH2 end of another. In other embodiments, AgRP can be in an acid form.

AgRP plays a role as an inverse agonist of the melanocortin receptors MC3R and MC4R. The term “AgRP peptide” in all instances includes salts thereof. In some embodiments, AgRP can be in an amide form, e.g., amidation of C-terminus —CO2H to form C(O)—NH2. In other embodiments, AgRP can be in an acid form.

The term “AgRP peptide” also includes shorter biologically active fragments of AgRP. A fragment is a portion of the parent sequence which is identical in sequence but shorter in length than the parent sequence and retain biological activity (i.e. inverse agonism). Fragments of AgRP polypeptides as well as variants thereof have also been described in Jackson, P. J. et al., Biochemistry 41, 7565-7572, which is incorporated by reference herein. For example AgRP (87-120) and AgRP(87-132) possess approximately the same MC3R and MC4R affinity as AgRP(83-132) and exhibit equivalent inverse agonism. Additional fragments of AgRP polypeptide have been described in Christine G. Joseph et al., Peptides 24 (2003), 263-270; which is incorporated by reference herein. Examples of fragments are AgRP(86-132) and monocyclic AgRP (109-118) as well as elongation thereof at the N- and/or C-terminus.

The term “AgRP polypeptides” also encompasses “AgRP mutant polypeptide” which are AgRP polypeptide in which a naturally occurring AgRP polypeptide sequence has been modified. Such modifications have been described in PCT application No. WO2013/006656, which is incorporated by reference herein.

AgRP (83-132)'s C-terminus can be either Amide or Acid: Ser-Ser-Arg-Arg-Cys-Val-Arg-Leu-His-Glu-Ser-Cys-Leu-Gly-Gln-Gln-Val-Pro-Cys-Cys-Asp-Pro-Cys-Ala-Thr-Cys-Tyr-Cys-Arg-Phe-Phe-Asn-Ala-Phe-Cys-Tyr-Cys- Arg-Lys-Leu-Gly-Thr-Ala-Met-Asn-Pro-Cys-Ser-Arg-Thr (C1&C4, C2&C6, C3&C9, C5&C10,C7&C8 Bridges)(SEQ ID NO:1).

The term “vector” is used to refer to any molecule (e.g., nucleic acid, plasmid, or virus) used to transfer coding information to a host cell.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “amino acid,” as used herein, refers to naturally occurring amino acids, unnatural amino acids, amino acid analogues and amino acid mimetics that function in a manner similar to the naturally occurring amino acids, all in their D and L stereoisomers if their structure allows such stereoisomeric forms Amino acids are referred to herein by either their name, their commonly known three letter symbols or by the one-letter symbols recommended by the IUPAC-IUB Biochemical Nomenclature Commission.

The term “naturally occurring” when used in connection with biological materials such as nucleic acid molecules, polypeptides, host cells, and the like, refers to materials that are found in nature and are not manipulated by man. Similarly, “non-naturally occurring” as used herein refers to a material that is not found in nature or that has been structurally modified or synthesized by man. When used in connection with nucleotides, the term “naturally occurring” refers to the bases adenine (A), cytosine (C), guanine (G), thymine (T), and uracil (U). When used in connection with amino acids, the term “naturally occurring” refers to the 20 conventional amino acids (i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y)), as well as selenocysteine, pyrrolysine (PYL), and pyrroline-carboxy-lysine (PCL).

The term “amino acid mimetics,” as used herein, refers to chemical compounds that have a structure that is different from the general chemical structure of an amino acid, but functions in a manner similar to a naturally occurring amino acid.

The term “biologically active variant” refers to any AgRP polypeptide analog or variant used in the invention that possesses an activity of wild-type (e.g., naturally-occurring) AgRP, such as the ability to stimulate appetite; decrease energy expenditure; and/or increase food intake, body weight, and/or fat mass. Polypeptide variants possessing a somewhat decreased level of activity relative to their wild-type versions can nonetheless be considered to be biologically active polypeptide variants, although ideally a biologically active polypeptide possesses similar or enhanced biological properties relative to its wild-type protein counterpart.

The terms “effective amount” and “therapeutically effective amount” each refer to the amount of an AgRP, analog or peptide used to support an observable level of one or more biological activities of the wild-type AgRP, such as the ability to improve appetite; and/or to increase food intake, body weight, and/or fat mass. For example, a “therapeutically-effective amount” administered to a patient exhibiting, suffering, or prone to suffer from wasting disorders such as cachexia (e.g., anorexia cachexia), is such an amount which causes an improvement in the pathological symptoms, disease progression, physiological conditions associated with or induces resistance to succumbing to the aforementioned disorders.

For the purposes of the present invention a “subject” or “patient” is preferably a human, but can also be an animal, more specifically, a companion animal (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “physiologically acceptable carrier” as used herein refers to one or more formulation materials suitable for accomplishing or enhancing the delivery of AgRP, analog or peptide.

The term “wasting disorders,” and terms similarly used herein, includes but is not limited to anorexia cachexia, anorexia of the aged, anorexia nervosa, cachexia associated with cancer, cachexia associated with AIDS, cachexia associated with heart failure, cachexia associated with cystic fibrosis, cachexia associated with rheumatoid arthritis, cachexia associated with kidney disease, cachexia associated with COPD, cachexia associated with ALS, cachexia associated with renal failure or cachexia associated, and other disorders associated with aberrant appetite, fat mass, energy balance, and/or involuntary weight loss.

As used herein, the singular forms “a,” “an” and “the” include plural references unless the content clearly dictates otherwise. As used herein, the term “about” refers to +/−20%, +/−10%, or +/−5% of a value.

The terms “polypeptide” and “protein”, are used interchangeably and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous signal sequences, with or without N-terminal methionine residues; immunologically tagged proteins; and the like.

The terms “individual,” “subject,” “host,” and “patient,” are used interchangeably and refer to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, the term “modulating” refers to a change in the quality or quantity of a gene, protein, or any molecule that is inside, outside, or on the surface of a cell. The change can be an increase or decrease in expression or level of the molecule. The term “modulates” also includes changing the quality or quantity of a biological function/activity including, without limitation, the ability to increase body weight, appetite, and/or food intake.

As used herein, the term “modulator” refers to a composition that modulates one or more physiological or biochemical events associated with a wasting disorder, such as cachexia anorexia. Said events include but are not limited to the ability to reduce signaling through the hypothalamic melanocortin receptors (MCRs) 3 and 4; to stimulate feeding and decrease energy expenditure; and to increase body weight, appetite, and/or food intake.

A “gene product” is a biopolymeric product that is expressed or produced by a gene. A gene product may be, for example, an unspliced RNA, an mRNA, a splice variant mRNA, a polypeptide, a post-translationally modified polypeptide, a splice variant polypeptide etc. Also encompassed by this term are biopolymeric products that are made using an RNA gene product as a template (i.e., cDNA of the RNA). A gene product may be made enzymatically, recombinantly, chemically, or within a cell to which the gene is native. In some embodiments, if the gene product is proteinaceous, it exhibits a biological activity. In some embodiments, if the gene product is a nucleic acid, it can be translated into a proteinaceous gene product that exhibits a biological activity.

AgRP activity can be assessed by means including, without limitation, assaying body composition, body weight, energy expenditure, core body temperature, appetite, blood glucose, insulin, triglyceride, cholesterol levels or functional status in a subject, assessing AgRP polypeptide levels, or by assessing AgRP transcription levels. Comparisons of AgRP activity can also be accomplished by, e.g., measuring levels of an AgRP downstream biomarker, and measuring increases in AgRP signaling.

AgRP activity can also be assessed by measuring interactions between AgRP and an AgRP receptor; e.g., melanocortin receptors (MCRs) 3 and 4. In some embodiments modulation of AgRP activity can cause modulation of an AgRP-related phenotype.

An “AgRP downstream biomarker,” as used herein, is a gene or gene product, or measurable indicia of a signaling pathway, gene or gene product. In some embodiments, a gene or activity that is a downstream marker of AgRP exhibits an altered level of expression. In some embodiments, an activity of the downstream marker is altered in the presence of an AgRP modulator. In some embodiments, the downstream markers exhibit altered levels of expression when AgRP is perturbed with an AgRP modulator of the present invention.

As used herein, the term “intranasal administration,” “IN administration,” and the like refer to administration via nasal devices, including, devices optimized for nose to brain delivery, e.g., travel along nasal olfactory and trigeminal nerves to CNS.

As used herein, the term “N-terminus” refers to at least the first 10 amino acids of a protein. As used herein, the term “C-terminus” refers to at least the last 10 amino acids of a protein.

The term “domain” as used herein refers to a structural part of a biomolecule that contributes to a known or suspected function of the biomolecule. Domains may be co-extensive with regions or portions thereof and may also incorporate a portion of a biomolecule that is distinct from a particular region, in addition to all or part of that region.

The term “region” refers to a physically contiguous portion of the primary structure of a biomolecule. In the case of proteins, a region is defined by a contiguous portion of the amino acid sequence of that protein. In some embodiments a “region” is associated with a function of the biomolecule.

The term “fragment” as used herein refers to a physically contiguous portion of the primary structure of a biomolecule. In the case of proteins, a portion is defined by a contiguous portion of the amino acid sequence of that protein and refers to at least 3-5 amino acids, at least 8-10 amino acids, at least 11-15 amino acids, at least 17-24 amino acids, at least 25-30 amino acids, and at least 30-45 amino acids. In the case of oligonucleotides, a portion is defined by a contiguous portion of the nucleic acid sequence of that oligonucleotide and refers to at least 9-15 nucleotides, at least 18-30 nucleotides, at least 33-45 nucleotides, at least 48-72 nucleotides, at least 75-90 nucleotides, and at least 90-130 nucleotides. In some embodiments, portions of biomolecules have a biological activity. In the context of the present invention, AgRP polypeptide fragments do not comprise the entire AgRP polypeptide sequence.

A “native sequence” polypeptide is one that has the same amino acid sequence as a polypeptide derived from nature. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other species.

As used herein, the phrase “homologous nucleotide sequence,” or “homologous amino acid sequence,” or variations thereof, refers to sequences characterized by a homology, at the nucleotide level or amino acid level, of at least a specified percentage and is used interchangeably with “sequence identity.” Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals.

Homologous nucleotide sequences also include, but are not limited to, naturally occurring allelic variations and mutations of the nucleotide sequences set forth herein. Homologous amino acid sequences include those amino acid sequences which contain conservative amino acid substitutions and polypeptides that have the same or similar binding and/or activity. In some embodiments, a nucleotide or amino acid sequence is homologous if it has at least 60%, 70%, 80%, 85%, 90%, 96%, or 98% identity. In some embodiments, a nucleotide or amino acid sequence is homologous if it has 1-10, 10-20, 20-30, 30-40, or 40-45 nucleotide/amino acid substitutions, additions, or deletions. In some embodiments, the homologous amino acid sequences have no more than 5 or no more than 3 conservative amino acid substitutes.

Percent homology or identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, homology between the probe and target is between about 75% to about 85%. In some embodiments, nucleic acids have nucleotides that are at least about 90%, about 96%, about 98%, and about 100% homologous to disclosed AgRP wild type sequences, or a portion thereof.

Homology may also be at the polypeptide level. In some embodiments, polypeptides are about 90%, about 96%, about 98%, and about 100% homologous to disclosed AgRP wild type sequences, or a portion thereof.

As used herein, the term “mixing” refers to the process of combining one or more compounds, cells, molecules, and the like together in the same area. This may be performed, for example, in a test tube, petri dish, or any container that allows the one or more compounds, cells, or molecules, to be intermingled.

As used herein, the term “substantially purified” refers to a compound (e.g., either a polynucleotide or a polypeptide) that is removed from its natural environment and is at least 60% free, at least 75% free, and at least 90% free from other components with which it is naturally associated.

It must be noted that as used herein and in the appended claims, the singular forms “a”, “an” and “the” include plural referents unless the context clearly dictates otherwise. Thus, for example, reference to “the conjugate” includes reference to one or more conjugates; reference to “the polypeptide” includes reference to one or more polypeptides; and so forth.

The term alkyl refers to a fully saturated branched or unbranched (or straight chain or linear) hydrocarbon moiety, comprising 1 to 30 carbon atoms. Preferably the alkyl comprises 5 to 20 carbon atoms, and more preferably 10 to 15 carbon atoms. C₁₀₋₁₅alkyl refers to an alkyl chain comprising 10 to 15 carbons.

The term “alkenyl” refers to a branched or unbranched hydrocarbon having at least one carbon-carbon double bond. The term “C₂₋₃₀-alkynyl” refers to a hydrocarbon having two to seven carbon atoms and comprising at least one carbon-carbon triple

The term “alkynyl” refers to a branched or unbranched hydrocarbon having at least one carbon-carbon triple bond. The term “C₂₋₃₀-alkynyl” refers to a hydrocarbon having two to seven carbon atoms and comprising at least one carbon-carbon triple bond.

The term aryl refers to monocyclic or bicyclic aromatic hydrocarbon groups having 6-10 carbon atoms in the ring portion. Representative examples of aryl are phenyl or naphthyl.

The term heteroaryl includes monocyclic or bicyclic heteroaryl, containing from 5-10 ring members selected from carbon atoms and 1 to 5 heteroatoms, and each heteroatoms is independently selected from O, N or S wherein S and N may be oxidized to various oxidation states. For bicyclic heteroaryl system, the system is fully aromatic (i.e. all rings are aromatic).

The term cycloalkyl refers to saturated or unsaturated but non-aromatic monocyclic, bicyclic or tricyclic hydrocarbon groups of 3-12 carbon atoms, preferably 3-8, or 3-7 carbon atoms. For bicyclic, and tricyclic cycloalkyl system, all rings are non-aromatic. For example, cycloalkyl encompasses cycloalkenyl and cycloalkynyl. The term “cycloalkenyl” refers to a bicyclic or tricyclic hydrocarbon group of 3-12 carbon atoms, having at least one carbon-carbon double bond. The term “cycloalkynyl” refers to a bicyclic or tricyclic hydrocarbon group of 3-12 carbon atoms, having at least one carbon-carbon triple bond.

The term heterocyclyl refers to a saturated or unsaturated non-aromatic (partially unsaturated but non-aromatic) monocyclic, bicyclic or tricyclic ring system which contains at least one heteroatom selected from O, S and N, where the N and S can also optionally be oxidized to various oxidation states. In one embodiment, heterocyclyl moiety represents a saturated monocyclic ring containing from 5-7 ring atoms and optionally containing a further heteroatom, selected from O, S or N. The heterocyclic ring may be substituted with alkyl, halo, oxo, alkoxy, haloalkyl, haloalkoxy. In other embodiment, heterocyclyl is di- or tricyclic. For polycyclic system, some ring may be aromatic and fused to saturated or partially saturated ring or rings. The overall fused system is not fully aromatic. For example, a heterocyclic ring system can be an aromatic heteroaryl ring fused with saturated or partially saturated cycloalkyl ring system.

The term “conjugate” is intended to refer to the entity formed as a result of a covalent attachment of biomolecule and a fatty acid moiety, via a linker.

Biomolecule or Biologically Active Molecule:

As used herein the term biomolecule or biologically active molecule includes, but is not limited to, antibodies (e.g., monoclonal, chimeric, humanized, nanobodies, and fragments thereof etc.), cholesterol, hormones, peptides, proteins, chemotherapeutics and other types of antineoplastic agents, low molecular weight drugs, vitamins, co-factors, nucleosides, nucleotides, oligonucleotides, enzymatic nucleic acids, antisense nucleic acids, triplex forming oligonucleotides, antisense DNA or RNA compositions, chimeric DNA:RNA compositions, allozymes, aptamers, ribozyme, decoys and analogs thereof, plasmids and other types of expression vectors, and small nucleic acid molecules, RNAi agents, short interfering nucleic acid (siNA), messenger ribonucleic acid” (messenger RNA, mRNA), short interfering RNA (siRNA), double-stranded RNA (dsRNA), micro-RNA (miRNA), and short hairpin RNA (shRNA) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), aiRNA (assymetrical interfering RNA), and siRNA with 1, 2 or more mismatches between the sense and anti-sense strand to relevant cells and/or tissues, such as in a cell culture, subject or organism. Such compounds may be purified or partially purified, and may be naturally occurring or synthetic, and may be chemically modified.

In one embodiment the biomolecule (or biologically active molecule) is a polypeptide, peptide, proteins or a RNAi agent, short interfering nucleic acid (siNA), short interfering RNA (siRNA), double-stranded RNA (dsRNA), micro-RNA (miRNA), or a short hairpin RNA (shRNA) molecule.

The term “pharmaceutically acceptable carrier” refers to a carrier for administration of a therapeutic agent, such as polypeptides, genes, or other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

The pharmaceutical compositions of the AgRP proteins, AgRP analogs and peptides of the methods of the present invention may be administered by any means that achieve the generally intended purpose: to treat for wasting disorders such as wasting disorders such as cachexia (e.g., anorexia cachexia, also known as anorexia of the elderly; and cachexia associated with cancer, AIDS, heart failure, kidney disease, ALS, and COPD) or anorexia nervosa, and in reducing the mortality and morbidity of critically ill patients.

The pharmaceutical compositions of the AgRP proteins, AgRP analogs and peptides of the methods of the present invention may also be administered by any means that achieve the generally intended purpose of treating one or more of the following: Cushing's syndrome, Hypercortisolism, the ectopic ACTH syndrome, the change in adrenocortical mass, primary pigmented nodular adrenocortical disease (PPNAD) Carney complex (CNC), the cortisol-induced mineralocorticoid excess, conditions associated with post-traumatic stress disorder, hirsutism, thin skin, myopathy, osteoporosis, increased tissue fragility, poor wound healing, hypertension, diabetes mellitus, low serum potassium, and low eosinophils and lymphopenia.

The required AgRP dosage administered will be dependent upon the age, health, and weight of the recipient, kind of concurrent treatment, if any, frequency of treatment, and the nature of the effect desired. Compositions within the scope of the invention include all compositions wherein an AgRP protein, AgRP analog or AgRP peptides is present in an amount that is effective to achieve the desired medical effect for treatment of the wasting disorders listed herein, including but not limited to wasting disorders such as cachexia (e.g., anorexia cachexia, also known as anorexia of the elderly; and cachexia associated with cancer and AIDS). While individual needs may vary from one patient to another, the determination of the optimal ranges of effective amounts of all of the components is within the ability of the clinician of ordinary skill.

The AgRP proteins, AgRP analogs and AgRP peptides of the methods of the present invention can be formulated according to known methods to prepare pharmaceutically useful compositions. A desired formulation would be one that is a stable lyophilized product that is reconstituted with an appropriate diluent or an aqueous solution of high purity with optional pharmaceutically acceptable carriers, preservatives, excipients or stabilizers [Remington's Pharmaceutical Sciences 16th edition (1980)]. The variants of the present invention may be combined with a pharmaceutically acceptable buffer, and the pH adjusted to provide acceptable stability, and a pH acceptable for administration.

For parenteral administration, in one embodiment, the AgRP proteins, AgRP analogs and AgRP peptides are formulated generally by mixing one or more of them at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. Preferably, one or more pharmaceutically acceptable anti-microbial agents may be added. Phenol, m-cresol, and benzyl alcohol are preferred pharmaceutically acceptable anti-microbial agents.

Optionally, one or more pharmaceutically acceptable salts may be added to adjust the ionic strength or tonicity. One or more excipients may be added to further adjust the isotonicity of the formulation. Glycerin, sodium chloride, and mannitol are examples of isotonicity adjusting excipients.

The therapeutic compositions employed by the methods of the present invention, and/or comprising the AgRP, AgRP analog, or AgRP peptides of the invention, may be used as a regularly intranasally-administered (e.g., before each meal, bidaily (BID), daily, twice a week, weekly, biweekly, or monthly) agent, either alone or in combination with other agents known to stimulate weight or skeletal muscle gain in humans such as Megestrol acetate; olanzapine; an antibody directed to the myostatin/activinII receptor, such as BYM338 (bimagrumab), that stimulates skeletal muscle growth (Lach-Trifilieff et al., (2014) Mol. Cell. Biol. 34(4): 606-618) under conditions of adequate caloric intake; Ghrelin (Wren et al. (2001) JCEM 86(12); or proposed stimulating agents such as Neuropeptide Y, H3 Relaxin (McGowan et al, (2005) Endocrinology 146(8):3295-3300) (Schwarts M W et al (2000) Nature 404, 661-671.

Those skilled in the art can readily optimize pharmaceutically effective dosages and administration regimens for therapeutic compositions comprising AgRP, AgRP analog or peptides, as determined by good medical practice and the clinical condition of the individual patient. A typical dose range for the AgRP, AgRP analog or AgRP peptides of the methods of the present invention will range from about 0.1 mg per day to about 40 mg per day (or about 0.7 mg per week to about 280 mg per week administered daily, once per week, bi-weekly, or monthly for an adult. Preferably, the dosage ranges from about 1.0 mg per day to about 10 mg per day (or about 0.7 mg per week to about 70 mg per week administered once per day, week, bi-weekly, or monthly).

The appropriate dose of an AgRP, AgRP analog or AgRP peptides administered is useful for treating the wasting disorders listed herein, including but not limited to cachexia (e.g., anorexia cachexia, and cachexia associated with cancer and AIDS).

Having now described the present invention in detail, the same will be more clearly understood by reference to the following examples, which are included herewith for purposes of illustration only and are not intended to be limiting of the invention.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); and Sambrook et al., Molecular Cloning: A Laboratory Manual (2nd Edition, 1989).

### Therapeutic Compositions of AgRP and Administration Thereof

Any polynucleotides utilized by the present methods of the invention will be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences. The AgRP variant can be expressed in mammalian cells, insect, yeast, bacterial or other cells under the control of appropriate promoters. Cell free translation systems can also be employed to produce such proteins using RNAs derived from DNA constructs of the present invention.

E. coli is a prokaryotic host useful particularly for cloning the polynucleotides of the present invention. Other microbial hosts suitable for use include Bacillus subtilis, Salmonella typhimurium, and various species of Serratia, Pseudomonas, Streptococcus, and Staphylococcus, although others may also be employed as a matter of choice. In these prokaryotic hosts, one can also make expression vectors, which will typically contain expression control sequences compatible with the host cell (e.g., an origin of replication). In addition, any of a number of well-known promoters may be present, such as the lactose promoter system, a tryptophan (Trp) promoter system, a beta-lactamase promoter system, or a promoter system from phages lambda or T7. The promoters will typically control expression, optionally with an operator sequence, and have ribosome binding site sequences and the like, for initiating and completing transcription and translation.

One skilled in the art of expression of proteins will recognize that methionine or methionine-arginine sequence can be introduced at the N-terminus of the mature sequence for expression in E. coli and are contemplated within the context of this invention. Thus, unless otherwise noted, compositions of the present invention expressed in E. coli have a methionine sequence introduced at the N-terminus.

Other microbes, such as yeast or fungi, may also be used for expression. Pichia pastoris, Saccharomyces cerevisiae, Schizosaccharomyces pombe, and Pichia angusta are examples of preferred yeast hosts, with suitable vectors having expression control sequences, such as promoters, including 3-phosphoglycerate kinase or other glycolytic enzymes, and an origin of replication, termination sequences and the like as desired. Aspergillus niger, Trichoderma reesei; and Schizophyllum commune, are examples of fungi hosts, although others may also be employed as a matter of choice.

Mammalian tissue cell culture may also be used to express and produce the polypeptides of the present invention. Eukaryotic cells are actually preferred, because a number of suitable host cell lines capable of secreting intact variants have been developed in the art, and include the CHO cell lines, various COS cell lines, NSO cells, Syrian Hamster Ovary cell lines, HeLa cells, or human embryonic kidney cell lines (i.e. HEK293, HEK293EBNA).

Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Preferred expression control sequences are promoters derived from SV40, adenovirus, bovine papilloma virus, cytomegalovirus, Raus sarcoma virus, and the like. Preferred polyadenylation sites include sequences derived from SV40 and bovine growth hormone.

The vectors containing the polynucleotide sequences of interest (e.g., that encode the compositions of the present invention and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts.

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used for the compositions of the present invention.

The proteins, polypeptides, and/or peptides utilized by the present methods of the invention should be formulated and dosed in a fashion consistent with good medical practice, taking into account the clinical condition of the patient, the site of delivery of the protein compositions, the method of administration, the scheduling of administration, and other factors known to practitioners. The “therapeutically effective amount” of the compositions of the present invention for purposes herein is thus determined by such considerations.

The pharmaceutical compositions utilized by the present methods of the proteins of the present invention may be administered by any means that achieve the generally intended purpose: to treat wasting disorders such as cachexia (e.g., anorexia cachexia, also known as anorexia of the elderly; and cachexia associated with cancer, AIDS, heart failure, kidney disease, ALS, and COPD) or anorexia nervosa, and other disorders associated with aberrant appetite, fat mass, energy balance, and/or involuntary weight loss, and to reduce the mortality and morbidity of critically ill patients. Non-limiting permissible means of administration include, for example, by inhalation, suppository or application to mucosal tissue such as by lavage to vaginal, rectal, urethral, buccal and sublingual tissue, orally, nasally, topically, intranasally, intraperitoneally, parenterally, intravenously, intramuscularly, intrasternally, by intraarticular injection, intralymphatically, interstitially, intra-arterially, subcutaneously, intrasynovial, transepithelial, and transdermally.

Preferred means of administration, as further described herein, are nasal and intranasal means.

In some embodiments, the pharmaceutical compositions are administered by lavage, orally or inter-arterially. Other suitable methods of introduction can also include rechargeable or biodegradable devices and slow or sustained release polymeric devices. The pharmaceutical compositions of this invention can also be administered as part of a combinatorial therapy with other known therapeutic agents, especially those that improve wasting disorders

The dosage administered will be dependent upon the age, health, and weight of the recipient, kind of concurrent treatment, if any, frequency of treatment, and the nature of the effect desired. Compositions within the scope of the invention include all compositions wherein an AgRP variant is present in an amount that is effective to achieve the desired medical effect for treatment of cachexia (e.g., anorexia cachexia, also known as anorexia of the elderly; and cachexia associated with cancer, AIDS, heart failure, kidney disease, ALS, and COPD) anorexia nervosa, and other wasting disorders, and other wasting disorders. While individual needs may vary from one patient to another, the determination of the optimal ranges of effective amounts of all of the components is within the ability of the clinician of ordinary skill.

Any compositions utilized by the present methods of the present invention can be formulated according to known methods to prepare pharmaceutically useful compositions. A desired formulation would be one that is a stable lyophilized product that is reconstituted with an appropriate diluent or an aqueous solution of high purity with optional pharmaceutically acceptable carriers, preservatives, excipients or stabilizers [Remington's Pharmaceutical Sciences 16th edition (1980)]. The compositions of the present invention may be combined with a pharmaceutically acceptable buffer, and the pH adjusted to provide acceptable stability, and a pH acceptable for administration.

For parenteral administration, in one embodiment, the compositions of the present invention are formulated generally by mixing one or more of them at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. Preferably, one or more pharmaceutically acceptable anti-microbial agents may be added. Phenol, m-cresol, and benzyl alcohol are preferred pharmaceutically acceptable anti-microbial agents.

Optionally, one or more pharmaceutically acceptable salts may be added to adjust the ionic strength or tonicity. One or more excipients may be added to further adjust the tonicity of the formulation. Glycerin, sodium chloride, and mannitol are examples of an tonicity adjusting excipient.

Those skilled in the art can readily optimize pharmaceutically effective dosages and administration regimens for therapeutic compositions comprising compositions of the present invention, as determined by good medical practice and the clinical condition of the individual patient. A typical dose range for the compositions of the present invention will range from about 0.01 mg per day to about 150 mg per day (or about 0.07 mg per week to about 1050 mg per week) and can be administered before each meal, bidaily (BID), daily, twice a week, once per week, bi-weekly, or monthly for an adult. Preferably, the dosage ranges from about 0.1 mg per day to about 150 mg per day (or about 0.7 mg per week to about 1050 mg per week) and can be administered before each meal, bidaily (BID), daily, twice a week, once per week, bi-weekly, or monthly for an adult. Most preferably, the dosage is about 1-5 mg/day to about 150 mg per day (or about 7 mg per week to about 35 mg per week) and can be administered before each meal, bidaily (BID), daily, twice a week, once per week, bi-weekly, or monthly for an adult. The appropriate dose of an AgRP, AgRP analogs, or peptides administered will result in lowering blood glucose levels and increasing energy expenditure by faster and more efficient glucose utilization, and thus is useful for treating the wasting disorders listed herein, including but not limited to cachexia (e.g., anorexia cachexia, also known as anorexia of the elderly; and cachexia associated with cancer, AIDS, heart failure, kidney disease, and COPD) anorexia nervosa, and other disorders associated with aberrant appetite, fat mass, energy balance, and/or involuntary weight loss.

Methods of treatment using therapeutic compositions comprising AgRP proteins, AgRP variants and peptides are within the scope of the present invention. Such pharmaceutical compositions can comprise a therapeutically effective amount of an AgRP protein, variant or peptide in admixture with a pharmaceutically or physiologically acceptable formulation agent selected for suitability with the mode of administration.

Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, acetate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such asacetate), preservatives (such as benzalkonium chloride, benzoic acid, salicylic acid, thimerosal, phenethyl alcohol, methylparaben, propylparaben, or chlorhexidine, sorbic acid), solvents (such as glycerin, propylene glycol, or PEG), sugar alcohols (such as mannitol or sorbitol), suspending agents, surfactants or wetting agents (such as pluronics; PEG; sorbitan esters; polysorbates such as polysorbate 20 or polysorbate 80; triton; trimethamine; lecithin; cholesterol or tyloxapal), stability enhancing agents (such as sucrose or sorbitol), tonicity enhancing agents (such as alkali metal halides; preferably sodium or potassium chloride; or mannitol sorbitol), delivery vehicles, diluents, excipients and/or pharmaceutical adjuvants (see, e.g., Remington's Pharmaceutical Sciences (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage (see, e.g., Remington's Pharmaceutical Sciences, supra). Such compositions can influence the physical state, stability, rate of in vivo release, and rate of in vivo clearance of the AgRP protein, variant or peptide.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the present invention, AgRP protein, variant or peptide compositions can be prepared for storage by mixing the selected composition having the desired degree of purity with optional formulation agents (Remington's Pharmaceutical Sciences, supra) in the form of a lyophilized cake or an aqueous solution. Further, the AgRP protein, variant or peptide product can be formulated as a lyophilizate using appropriate excipients such as sucrose.

The AgRP protein, variant or peptide pharmaceutical compositions can be selected for parenteral delivery, and are most preferably selected for intranasal delivery. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

In one embodiment, a pharmaceutical composition can be formulated intranasal delivery. For example, a powder formulation is desired in order to allow the AgRP protein, variant or peptide to get better adherence into the mucosal membrane, such that it would not be inhaled. An example of said formulation is a dry powder for intranasal delivery. AgRP protein, variant or peptide intranasal solutions can also be formulated with a propellant for aerosol delivery. In yet another embodiment, solutions can be nebulized.

Another pharmaceutical composition can involve an effective quantity of AgRP protein or peptides in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional AgRP protein, variant or peptide pharmaceutical compositions will be evident to those skilled in the art, including formulations involving AgRP protein, variant or peptides in sustained- or controlled-delivery formulations. Techniques for formulating a variety of other sustained- or controlled-delivery means, such as liposome carriers, and bio-erodible microparticles or porous beads, are also known to those skilled in the art (see, e.g., International Publication No. WO 93/15722, which describes the controlled release of porous polymeric microparticles for the delivery of pharmaceutical compositions, and Wischke & Schwendeman, 2008, Int. J Pharm. 364: 298-327, and Freiberg & Zhu, 2004, Int. J Pharm. 282: 1-18, which discuss microsphere/microparticle preparation and use).

Additional examples of sustained-release preparations include semipermeable polymer matrices in the form of shaped articles, e.g. films, or microcapsules. Sustained release matrices can include polyesters, hydrogels, polylactides (U.S. Pat. No. 3,773,919 and European Patent No. 0 058 481), copolymers of L-glutamic acid and gamma ethyl-L-glutamate (Sidman et al., 1983, Biopolymers 22: 547-56), poly(2-hydroxyethyl-methacrylate) (Langer et al., 1981, J. Biomed. Mater. Res. 15: 167-277 and Langer, 1982, Chem. Tech. 12: 98-105), ethylene vinyl acetate (Langer et al., supra) or poly-D-3-hydroxybutyric acid (European Patent No. 0 133 988). Sustained-release compositions can also include liposomes, which can be prepared by any of several methods known in the art. See, e.g., Epstein et al., 1985, Proc. Natl. Acad. Sci. U.S.A. 82: 3688-92; and European Patent Nos. 0 036 676, 0 088 046, and 0 143 949.

The AgRP or peptide pharmaceutical composition to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a specific embodiment, the present invention is directed to kits for producing a single-dose administration unit. The kits can each contain both a first container having a dried protein and a second container having an aqueous formulation. Also included within the scope of this invention are kits containing single and multi-chambered pre-filled syringes (e.g., liquid syringes and lyosyringes).

The effective amount of an AgRP protein, variant or peptide pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the AgRP protein, variant or peptide is being used, the route of administration, and the size (body weight, body surface, or organ size) and condition (the age and general health) of the patient. Accordingly, the clinician can titer the dosage and modify the route of administration to obtain the optimal therapeutic effect. A typical dosage can range from about 0.1 mg to up to about 150 mg or more, depending on the factors mentioned above. In other embodiments, the daily dosage can range from 0.1 mg up to about 150 mg; or 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, 110 mg, 120 mg, 130 mg, 140 mg, up to about 150 mg.

The frequency of dosing will depend upon the pharmacodynamic parameters of the AgRP protein, variant or peptide in the formulation being used. Typically, a clinician will administer the composition until a dosage is reached that achieves the desired effect. The composition can therefore be administered as a single dose, as two or more doses (which may or may not contain the same amount of the desired molecule) over time. Further refinement of the appropriate dosage is routinely made by those of ordinary skill in the art and is within the ambit of tasks routinely performed by them. Appropriate dosages can be ascertained through use of appropriate dose-response data.

AgRP Biomolecules (Fatty-Acid Conjugates) Embodiments

In embodiment 1, the invention pertains to a conjugate comprising a biomolecule linked to an fatty acid moiety via a linker wherein the fatty acid moiety has the following Formulae A1, A2 or A3:

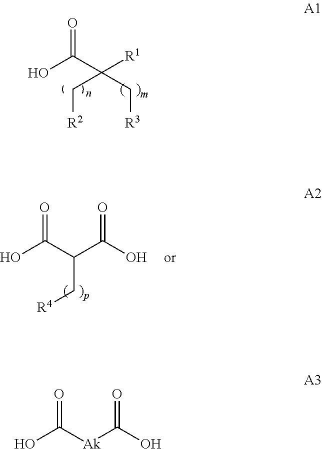

R¹ is CO₂H, H;

R², R³ and R⁴ are independently of each other H, OH, CO₂H, —CH═CH₂ or —C═CH;

Ak is a branched C₆-C₃₀alkylene;

n, m and p are independently of each other an integer between 6 and 30; or an amide, an ester or a pharmaceutically acceptable salt thereof.

In embodiment 1A, the invention pertains to a conjugate according to embodiment 1 wherein the fatty acid moiety is of Formula A1. In a particular aspect of this embodiment, the conjugate comprises a fatty acid moiety of Formula A1 wherein n and m are independently 8 to 20, preferably 10 to 16. In another aspect of this embodiment, the invention pertains to a conjugate according to embodiment 1 or 1A wherein the fatty acid moiety is of Formula A1 and wherein at least one of R2 and R3 is CO2H.

In embodiment 2, the invention pertains to a conjugate according to embodiment 1 or 1A, wherein the fatty acid moiety is selected from the following Formulae:

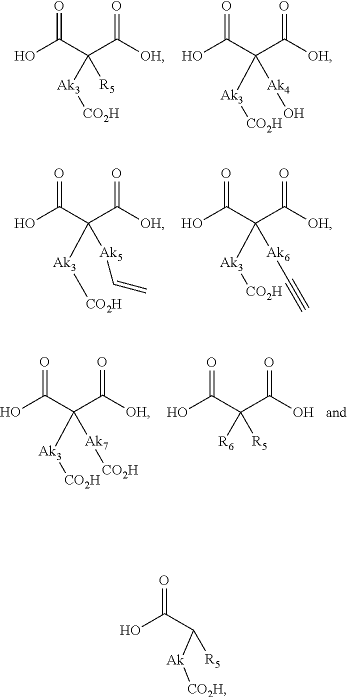

wherein Ak3, Ak4, Ak5, Ak6 and Ak7 are independently a (C₈₋₂₀)alkylene, R5 and R6 are independently (C₈₋₂₀)alkyl.

In embodiment 3, the invention pertains to a conjugate according to embodiment 1, 1A or 2 wherein the fatty acid moiety is selected from the following Formulae:

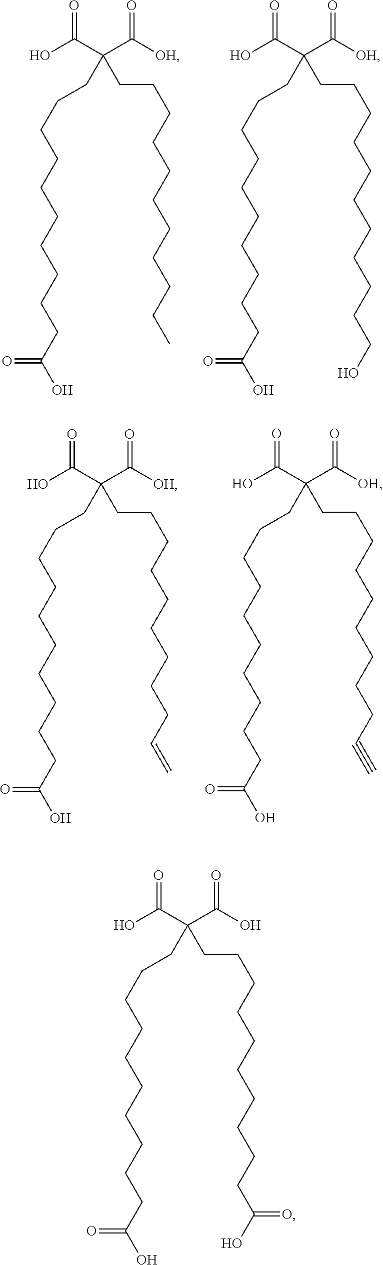

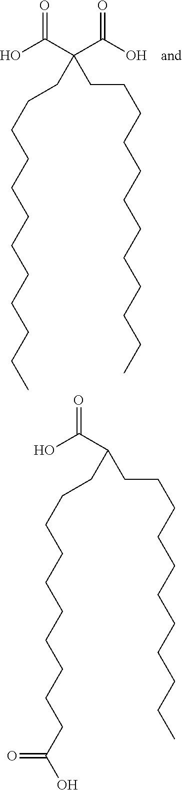 [SMILES: CCCCCCCCCCCCC(CCCCCCCCCCCC)(C(=O)O)C(=O)O ; CCCCCCCCCCCC(CCCCCCCCCCC(=O)O)C(=O)O]

In embodiment 3A, the invention pertains to a conjugate according to embodiment 1, 1A or 2 wherein the fatty acid moiety is selected from the following Formulae:

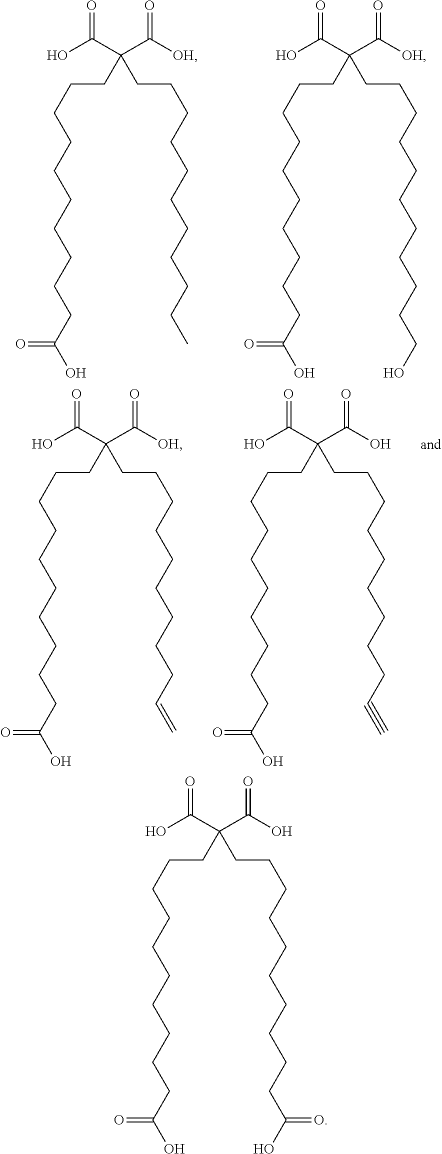

In embodiment 3B, the invention pertains to a conjugate according to embodiment 1 wherein the fatty acid moiety is of Formula A2 or A3. In a particular aspect of this embodiment, the conjugate comprises an fatty acid moeity of Formula A2 wherein p is 8 to 20, or a fatty acid moeity of Formula A3 wherein Ak is C₈₋₂₀alkylene.

In embodiment 3C, the invention pertains to a conjugate according to embodiment 1 or 3B wherein the fatty acid moeity is selected from the following Formulae:

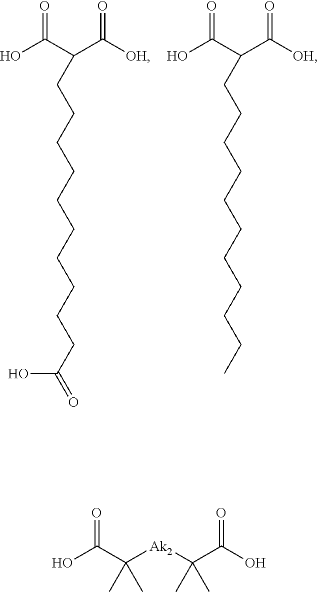

wherein Ak₂ is C₈₋₂₀alkylene.

In embodiment 4, the invention pertains to a conjugate according to any of the preceding embodiments wherein the linker comprise one or more alkyl groups, alkenyl groups, cycloalkyl groups, aryl groups, heteroaryl groups, heterocyclic groups, polyethylene glycol, one or more natural or unnatural amino acids, or combination thereof, wherein each of the alkyl, alkenyl, cycloalkyl, aryl, heteroaryl, heterocyclyl, polyethylene glycol and/or the natural or unnatural amino acids are optionally combined and linked together or linked to the biomolecule and/or to the fatty acid moiety via a chemical group selected from —C(O)O—, —OC(O)—, —NHC(O)—, —C(O)NH—, —O—, —NH—, —S—, —C(O)—, —OC(O)NH—, —NHC(O)—O—, ═NH—O—, ═NH—NH— or ═NH—N(alkyl)-.

In embodiment 5, the invention pertains to a conjugate according to any of the preceding embodiment, wherein the linker comprises an unbranched oligo ethylene glycol moiety of Formula:

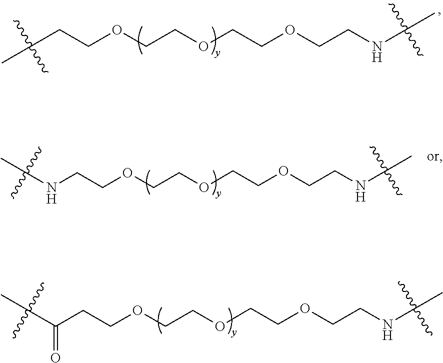

wherein y is 0 to 34.

In embodiment 6, the invention pertains to conjugate according to any of the preceding embodiments wherein the linker comprises (or further comprises) a heterocyclic moiety selected from the following Formulae:

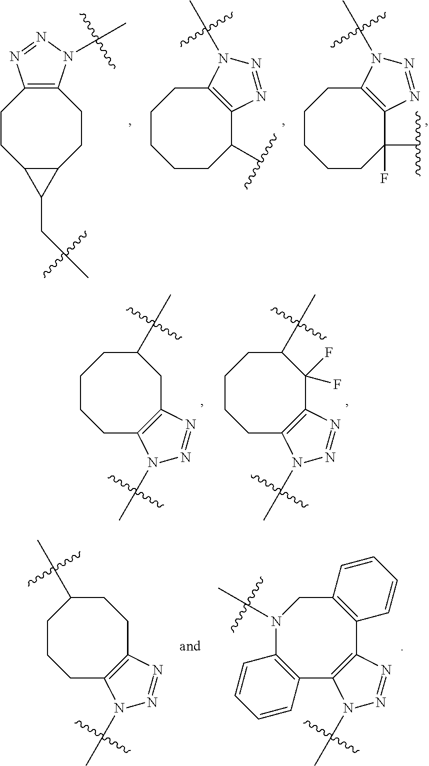

Such heterocyclyl containing linkers are obtained for example by azide-alkyne Huisgen cycloaddition, which more commonly known as click chemistry. More particularly, some of the heterocyclyl depicted supra result from the reaction of a cycloalkyne with an azide-containing moiety.

Cycloalkyne are readily available from commercial sources and can therefore be functionalized via cycloaddition with a moiety containing an azide functionality (e.g. a linker containing a terminal azide functionality). Examples of the use of cyclic alkyne click chemistry in protein labeling has been described in US 2009/0068738 which is herein incorporated by reference.

Non-limiting examples of cycloalkyne agents which can be used in Huisgen cycloaddition are:

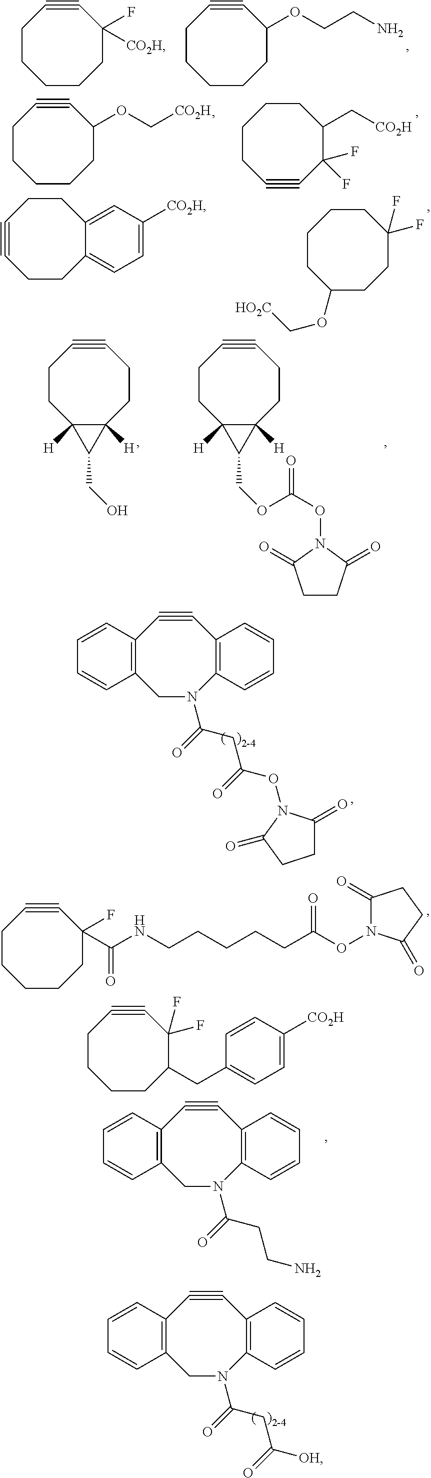

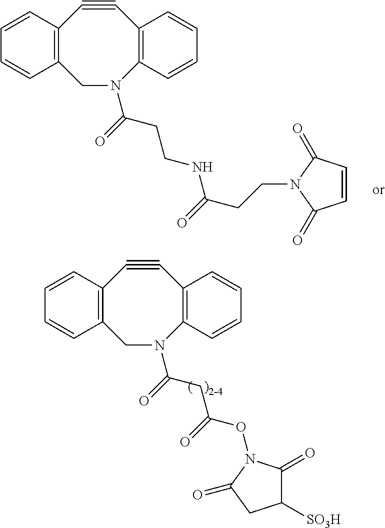

In embodiment 6A, the invention pertains to a conjugate according to any one of embodiments 1 to 5, wherein the linker comprises (or further comprises) a heterocyclyl selected from the following Formulae:

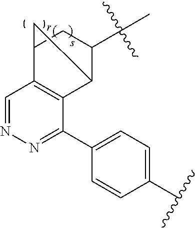

wherein r is an integer of 0 to 2 and s is an integer of 0 to 3.

Such heterocyclic linkers can be obtained via an aza [4+2] cycloaddition of an alkene, or preferably a strained alkene such as cycloalkane, with the following moiety:

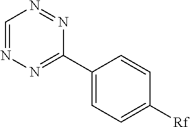

wherein Rf is for example —CH₂NH₂, —OH, —CH₂—CO₂H, —S—CH₂—CO₂H, —(O—CH₂)₄₋₆—C(O)—OH— or

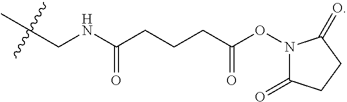 [SMILES: *CNC(=O)CCCC(=O)ON1C(=O)CCC1=O]

Such tetrazine moieties are readily available from commercial sources and can react with an alkene-containing moiety, for example a linker containing terminal alkene functionality.

In embodiment 6B, the invention pertains to a conjugate according to any one of embodiments 1 to 5 wherein the linker comprises (or further comprises) a heterocyclyl of Formula:

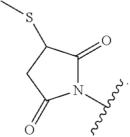 [SMILES: *N1C(=O)CC(SC)C1=O]

Such heterocyclic moiety can be obtained by reacting a maleimide with a thiol containing moiety, such as for example a linker containing a terminal thiol functionality.

These reagents which are readily available and/or commercially available are attached directly or via a linker as described supra to the peptide or polypeptide of interest. The alkyne, maleimide or tetrazine reactive groups are reacted with a functional group (azide, thiol and alkene respectively) which is present on the fatty acid moiety or on a linker-fatty acid construct (such as for example a PEG-fatty acid construct).

In embodiment 7, the invention pertains to a conjugate according to any of the preceding embodiments wherein the linker comprises or further comprises one or more amino acids independently selected from histidine, methionine, alanine, glutamine, asparagine and glycine. In one particular aspect of this embodiment, the linker comprises 1 to 6 amino acid selected from histidine, alanine and methionine.

It will be recognized that features specified in each embodiment may be combined with other specified features to provide further embodiments. In one embodiment, AgRP biomolecules (fatty-acid conjugates) embodiments pertains to a conjugate according to any one of the preceding embodiments wherein the biomolecule is a peptide or polypeptide.

The invention pertains to a conjugate according to any one of the preceding embodiments wherein the peptide or polypeptide an AgRP peptide. The invention pertains to a conjugate according to any one of the above embodiments wherein the biomolecule is an AgRP peptide. In a particular aspect of this embodiment, the AgRP peptide is AgRP(83-132) wherein the C-terminus is in the form of a -free CO2H or an amide thereof (e.g. —C(O)NH2).

Intranasal Delivery of Therapeutic Compositions of AgRP and Administration Thereof

In a preferred embodiment, AgRP is delivered via intranasal (IN) administration as a noninvasive method of brain delivery. Classically, administration of material into the brain requires an indwelling cannula placed either into a ventricle or directly into brain tissue, which is not a viable route for administration in humans to treat anorexia-cachexia. Central delivery of native AgRP to the brain has the potential to treat anorexia cachexia in humans via restoring normal appetite, and has been experimentally shown to increase appetite and food intake in lean rats and mice, as described herein.

The central nervous system (CNS) includes the brain, the brain stem, and the spinal cord. The CNS is isolated from the external world by several membranes that both cushion and protect the brain, the brain stem, and the spinal cord. For example, the membranes that form the blood-brain barrier (BBB) protect the brain from certain contents of the blood. The blood-cerebrospinal fluid barrier (BCSFB) protects other portions of the CNS from many chemicals and microbes. Traditional methods for delivering compounds to the CNS are typically invasive. For example, a pump implanted in the skull, such as an intracerebroventricular pump, can deliver a variety of compounds to the brain. However, implanting such a pump requires brain surgery, which can entail a variety of serious complications. Certain compounds, for example epidural painkillers, can be injected directly through the protective membrane into the CNS. However, such injection is impractical for most compounds.

Intranasal administration has traditionally focused on the distribution of drug solutions as a mist for topical delivery to the nasal epithelium. Because of the nasal cavity's easily accessed vascular bed, nasal administration of medications has focused the delivery of medications either locally to the nasal cavity or directly to the blood stream.

Much of the current brain research is focused on the enhancement of the drug being delivered to the brain by various formulations. The traditional approaches to improve uptake of compounds to the brain by formulation enhancement include (1) mucoadhesive formulations; 2) penetration enhancers; 3) liposomes; 4) vasoconstrictors; and 5) nanoparticles. Examples of various compounds which have enhanced formulations include various cytokines, for example, tumor necrosis factors, interleukins, and interferons discussed in U.S. Pat. No. 6,991,785 and growth and differentiation factor-5 (GDF-5) and related proteins discussed in US Publication No. 20100074959.

Targeting of drugs to the central nervous system (CNS) is a challenging task. A great number of drugs, including biotechnology products, are candidates for treatment of CNS diseases, but drug delivery is a problem for brain targeting. The BBB can allow small (about less than 500 Da), lipophilic molecules from the bloodstream to enter the CNS (Pardridge, Arch Neurol. 2002; 59:35-40). However, the BBB restricts access of large-molecules to the CNS (Pardride, NeuroRx. 2005 January; 2(1): 1-2. 2005).

Many larger therapeutic agents are prevented from reaching the brain for treating CNS disorders such as but not limited to Parkinson's disease, Alzheimer's disease, depression, stroke, and epilepsy (Pardridge, NeuroRx. 2005 January; 2(1): 3-14).

IN administration provides a non-invasive method of bypassing the blood brain barrier to deliver peptides, such as AgRP, to the brain. The human nasal cavity has a large absorptive surface area of ˜160 cm² with notable structural differences between primates and rodents. There are four types of epithelia in the nasal cavity, two of which (olfactory and respiratory) are most likely the sites of drug absorption for delivery to the brain. The olfactory nerve travels through the cribriform plate into the brain where it terminates on dendrites of olfactory bulb neurons, which project to deeper structures. The nasal respiratory epithelium lines approximately 80-90% of the nasal cavity in humans and, importantly, is innervated by branches of the trigeminal nerve, or fifth cranial nerve, which is the largest cranial nerve and mostly composed of somatic afferent fibers. After IN administration, peptides travel along the olfactory and trigeminal nerve components of the nasal epithelium to the olfactory bulb and brainstem, and this is followed by further dispersion to other brain nuclei (Campbell C, et al. (2012) Ther Deliv. April; 3(4):557-68).

In humans, IN insulin decreases food intake though hypothalamic action. Insulin is similar in size and structure to AgRP, and when administered intranasally, decreases food intake in a single test meal when compared to placebo. The decrease in food intake occurred at an IN dose that did not cause a measurable increase in systemic insulin concentrations, nor a reduction in plasma glucose, suggesting that insulin's anorexic effect was via direct action in the brain. (Jauch-Chara, et al. (2012) Diabetes. September; 61(9):2261-8).

A nasal spray is the designated route of administration to exploit the Nose-to-Brain pathway. For this, a simple formulation of the peptide in saline is foreseen. As nasal device a commercial nasal spray pump will be applied (e.g., Aptar, VP7 pump). This choice is based on the assumption that the device will deliver a certain amount of the API to the olfactory region of the nose although such devices are not optimized for this purpose.

Certain AgRP formulations work with conventional nasal spray pumps, and other formulations (e.g., powder, suspension or emulsion) work with nasal devices optimized for nose to brain delivery, e.g., travel along nasal olfactory nerves to CNS. In certain embodiments, these include simple formulations in saline (e.g., lyophilized for reconstitution), to be used in combination with a commercially available nasal spray pump (e.g., Aptar). Excipients may also be used to increase residence time on nasal mucosa (e.g. chitosan) by reducing clearance time. Said pumps enable AgRP delivery by bypassing the blood brain barrier, and makes the CNS accessible for peptides and molecules not able to pass the blood brain barrier. Said pumps require deposition of the drug (spray) to the olfactory region of the nasal cavity

Other potential devices for delivery include Optinoze (powered by exhalation of patient redirected into the nose, this leads to the closure of nose-throat channel, thus the drug is not inhaled); ViaNase, Kurve (suited for liquid formulations only; ability to deliver powder and liquid); and Impel Neuropharma POD (ability for brain to nose delivery, and ability to deliver powder and liquid).

### Therapeutic Uses of AgRP Proteins, AgRP Variants and Peptides

AgRP protein, variants or peptides can be used to treat, diagnose, ameliorate, or prevent a number of diseases, disorders, or conditions, including, but not limited to wasting disorders. In one embodiment, the wasting disorder to be treated is cachexia, e.g., anorexia cachexia. In another embodiment, the metabolic disorder is cachexia associated with cancer. In another embodiment, the metabolic disorder is cachexia associated with AIDS.

A disorder or condition such as anorexia cachexia can be treated by intranasally administering an AgRP protein, variant or peptide as described herein to a patient in need thereof in the amount of a therapeutically effective dose. In most situations, a desired dosage can be determined by a clinician, as described herein, and can represent a therapeutically effective dose of the AgRP protein, variant or peptide. It will be apparent to those of skill in the art that a therapeutically effective dose of AgRP protein, variant or peptide will depend, inter alia, upon the administration schedule, the unit dose of AgRP administered, whether the peptide or polypeptide is administered in combination with other therapeutic agents, the immune status and the health of the recipient.

The term “therapeutically effective dose,” as used herein, means that amount of AgRP protein, variant or peptide that elicits the biological or medicinal response in a tissue system, animal, or human being sought by a researcher, medical doctor, or other clinician, which includes alleviation of the symptoms of the disease or disorder being treated.

### Pharmaceutical Compositions

The methods of the present invention also employ pharmaceutical compositions comprising one or more of the AgRP or peptides or mutants thereof described herein and a pharmaceutically acceptable carrier, most preferably one suited for intranasal delivery. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

### Examples

### Example 1: Intranasally Delivered AgRP Stimulates Food Intake in Mice

The purpose of the following study was to determine if intranasally (IN) administered AgRP would stimulate food intake in C57BL mice.

IN administration may provide a non-invasive method for delivering therapeutic proteins directly to the central nervous system, bypassing the Blood-Brain Barrier (BBB), by transiting along olfactory- and trigeminal-associated extracellular pathways. Olfactory neurons are unique in that they interface directly with the nasal airway surface in the olfactory epithelium. These neurons then pass through the cribriform plate into the brain where they terminate at olfactory bulb neurons, which then project to deeper structures. The nasal respiratory epithelium is innervated by branches of the trigeminal nerve, or fifth cranial nerve, which may provide another conduit for molecules reaching the CNS (Lochhead and Thorne, 2012). AgRP is a large molecule that does not penetrate the brain with great efficiency when delivered systemically.

Research Design and Methods

26 week-old male C57BL mice were housed one per cage with normal chow diet and on a reversed light cycle (dark 11:00-23:00). Each mouse was given 25 μl of AgRP, formulated in saline, which had 57 μg of AgRP.

One day prior to treatment, 4-h food intake was measured and animals grouped to match group means of food intake. On the day of the study, food was removed at 9:00 am. Mice were anesthetized with isoflurane and IN dosed (25 μl) with saline or AgRP in saline. After dosing, the mice remained in the isoflurane chamber in a supine position for 3 min before being returned to their cages. A pre-weighed amount of food was given to the animals before the dark cycle. Food weight was recorded at 4 and 24 hours post dose.

Statistical Analysis

All data were analyzed using an unpaired t-test comparing saline and AgRP in saline. A p value of <0.05 or lower was taken to be statistically significant. All data were analyzed and plotted using GraphPad Prism6 software (San Diego, Calif.).

Results

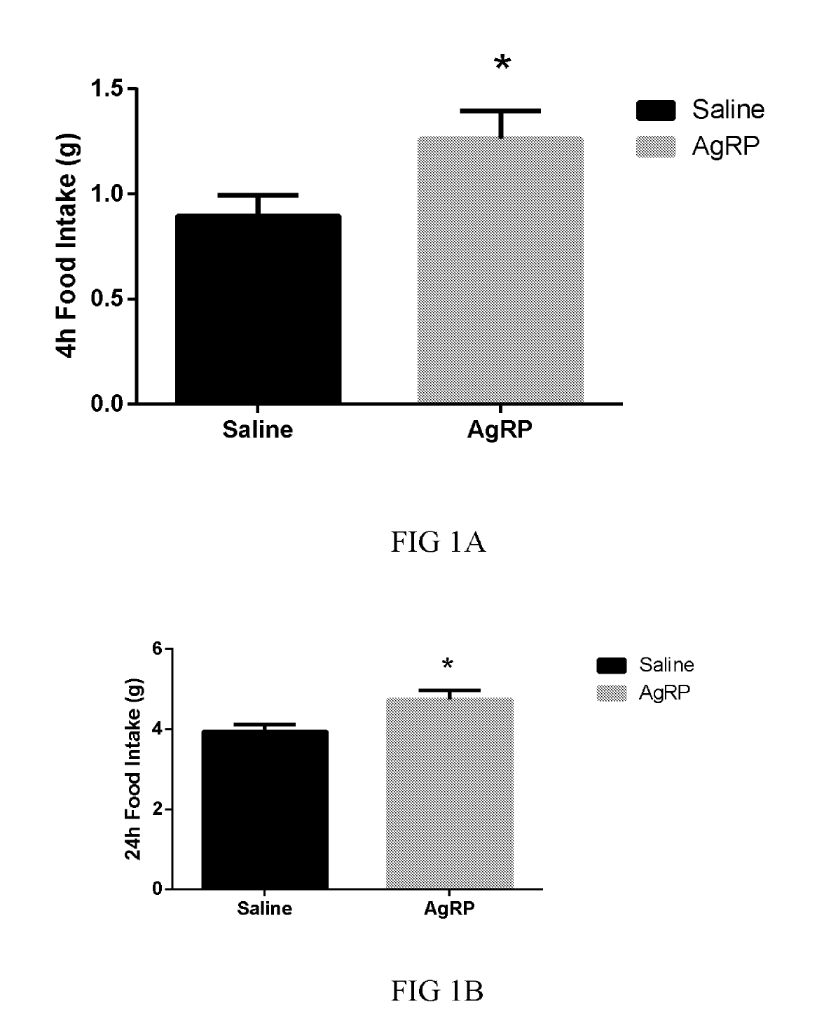

An unpaired t-test comparing saline and AgRP resulted in a significant effect at 4 h (FIG. 1A: t=2.2, p<0.05) and 24 h (FIG. 1B: t=2.6, p<0.05).

### Example 2: Subcutaneously Delivered AgRP does not Increase Food Intake in Mice

The purpose of the following study was to determine if subcutaneously (SC) administered AgRP would stimulate food intake in C57BL mice.

Research Design and Methods

Male C57BL mice were housed one per cage with normal chow diet and on a reversed light cycle (dark 11:00-23:00). Each mouse was given 25 μl of AgRP, formulated in saline, which had 57 μg of AgRP.

One day prior to treatment, 4-h food intake was measured and animals grouped to match group means of food intake. On the day of the study, animals were dosed via SC injection between 10:30 to 11:00 am. Pre-weighed food was provided to the mice prior to the dark cycle (11:00 am). At 4 and 24 h post dose food intake was measured.

Statistical Analysis

All data were analyzed using an unpaired t-test comparing PBS and AgRP in saline. A p value of <0.05 or lower was taken to be statistically significant. All data were analyzed and plotted using GraphPad Prism6 software (San Diego, Calif.).

Results

An unpaired t-test comparing PBS and AgRP did not resulted in a significant effect at 4 h (FIG. 2A: t=0.7, p>0.5) and 24 h (FIG. 2B: t=0.4, p>0.5).

### Example 3: Plasma Exposure Following Intranasal or Subcutaneous Delivery of AgRP in C57 Mice

The purpose of the following study was to determine if subcutaneously (SC) administered AgRP would stimulate food intake in C57BL mice.

Research Design and Methods

Male C57BL mice were housed one per cage with normal chow diet and housed in normal light cycle (light on 6:00-18:00). Each mouse was given 25 μl of AgRP, formulated in saline, which had 57 μg of AgRP.

10 min prior to the treatment, tail blood samples were taken and plasma (10 μl) was obtained. Mice receiving an IN dose of AgRP were anesthetized with isoflurane and IN dosed with 25 μl AgRP (57 μg). After being dosed, the mice remained in the isoflurane chamber for 2 min before placed back in their home cages. An alternate group of mice were SC dosed (114 μl) with AgRP (57 μg). Tail blood samples were taken at 2, 5, 10, 30 min and 1, 2, 4 and 24 h post dose. Plasma samples were aliquoted and stored at −80° C. until analysis.

Results

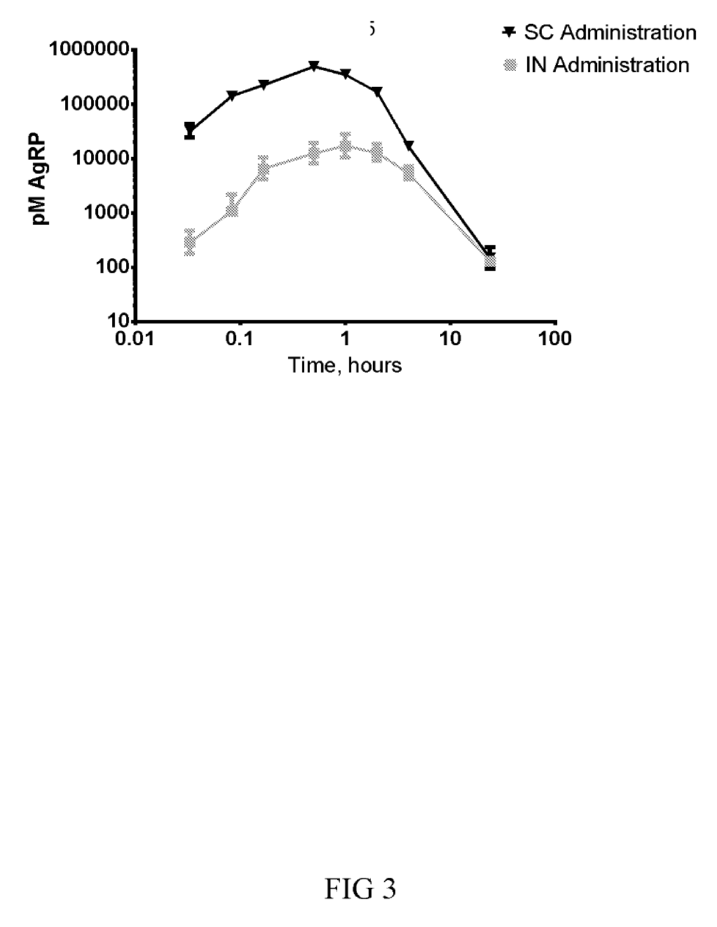

The Tmax for SC delivered AgRP was 0.5 h with a corresponding Cmax of 2810 ng/ml while for IN delivered AgRP had a Tmax of 1.0 h with a corresponding Cmax of 97.6 ng/ml (FIG. 3). Hence, the lack of increase in food intake in animals SC administered AgRP cannot be explained by the lack of plasma AgRP exposure.

### Example 4: Intranasally Delivered AgRP Stimulates Food Intake in Rats

The purpose of the following study was to determine if intranasal—(IN) administered AgRP would stimulate food intake in lean Sprague Dawley (SD) rats.

Research Design and Methods

8-12 week-old male SD rats were housed one per cage with a standard chow diet and normal light cycle. Each rat was given 50 μl of AgRP solution, formulated in saline, which had either 57 μg of AgRP or 171 μg of AgRP.

One and two days prior to dosing, 24 h food intake and body weight were measured. Animals were grouped based on body weight and food intake. On the day of the study, rats were briefly anesthetized with isoflurane and IN dosed (50 μl) with either saline or AgRP in saline. Animals were actively sniffing when compound was administered. After dosing, rats were returned to their home cages. A pre-weighed amount of food was given to the animals after dosing. Food weight was recorded at 4 and 24 hours post dose.

Statistical Analysis

All data were analyzed using an unpaired t-test comparing saline and AgRP. A p value of <0.05 or lower was taken to be statistically significant. All data were analyzed and plotted using GraphPad Prism6 software (San Diego, Calif.).

Results

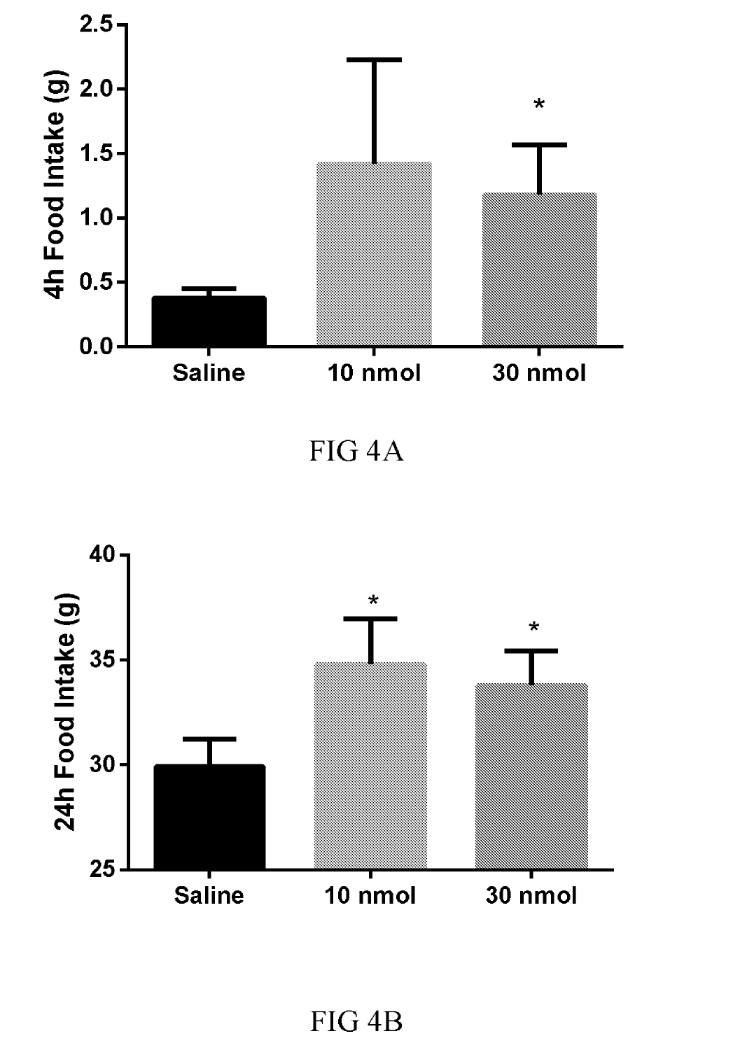

An unpaired t-test comparing saline and 171 μg AgRP resulted in a significant effect at 4 h (FIG. 4A: t=2.6, p<0.05) and both 57 μg and 171 μg AgRP elicited a significant increase at 24 h (FIG. 4B: t=2.1, and t=1.8 p<0.05).

### Example 5: Intranasally Delivered AgRP Elicits Hypothalamic AgRP Exposure in Rats

The purpose of the following study was to determine exposure of AgRP in lean SD rat hypothalamus after IN dosing of AgRP as compared to saline treated controls.

Research Design and Methods

Animals: Charles River SD rats were used. Rats were housed individually in on a standard chow diet with a 12 hour light cycle where the lights go out at 7 pm. Baseline body weights were used to appropriately balance treatment groups.

Administration of IN AgRP was delivered using a microsprayer aerosolizer (PennCentury, Wyndmoor, Pa.). First, rats were anesthetized with isoflurane, which is necessary to prevent unnecessary trauma to the delicate tissue in the nasal cavity. The animals were positioned so that they were lying on their backs. The tip of the microsprayer aerosolizer was placed into the skin flap/valve of one nare and used to navigate (or trace) the natural whorl of the skin flap/valve of the nares and inserted smoothly and with ease into the naris. The tip was inserted ˜3 cm (approximate distance from nares to cribriform plate). Once the tip was in place, saline or AgRP in saline was ejected from the microsprayer aerosolizer by pushing the plunger with a quick, sharp, firm and fast motion to produce a uniform puff of aerosol spray. The tip of the microsprayer aerosolizer was carefully removed from the animal's nasal cavity. 25 min after dosing, rats were given pentobarbital at 100 mg/kg ip injection and 5 min later, CSF and tail blood was collected. Tissues (nasal turbinates, olfactory bulb, hypothalamus, hind brain) were collected after whole body perfusion at 15 ml/min for 6 min with heparinized PBS (25 u/ml heparin in PBS).

Results

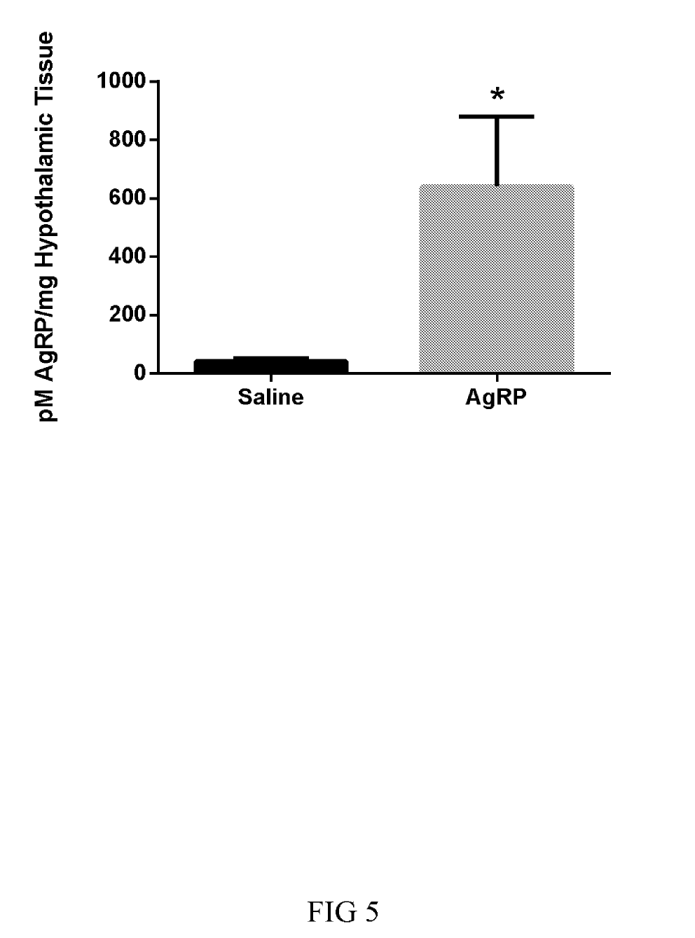

Animals dosed with AgRP had detectable levels of AgRP in the hypothalamus and other brain regions compared to saline treated animals (FIG. 5 and Table 1, BLOQ=below limit of quantitation). Due to only having two animals in the saline treated group, statistics were not performed.

TABLE 1

| | | pM AgRP/mg tissue | | | | |
|---|---|---|---|---|---|---|
| | | Nasal | Olfactory | | | |
| Animal NO. | Treatment | Turbinates | Bulb | Hypothalamus | Hindbrain | Cortex |
| 27 | AgRP | 43,168.7 | 2,479.6 | 330.0 | 2.8 | 3.3 |
| 35 | AgRP | 38,921.7 | 1,358.8 | 1,069.0 | 12.1 | 2.6 |
| 37 | AgRP | 7,360.6 | 9,683.2 | 1,320.0 | 8.1 | 25.4 |
| 39 | AgRP | 18.4 | 89.8 | 417.0 | 6.6 | 3.8 |
| 43 | AgRP | BLOQ | 7.8 | 71.0 | 1.4 | 0.9 |
| 30 | Saline | 27.8 | BLOQ | 34.0 | BLOQ | BLOQ |
| 32 | Saline | 17.3 | 4.7 | 51.5 | BLOQ | BLOQ |

### Example 5a and 5B: AgRP Biomolecules (Fatty-Acid Conjugates

AgRP(83-132)-FA Conjugates:

Example 5A: mono fatty acid conjugate of AgRP (AgRP+1FA) wherein the fatty acid is attached to the N-terminus of AgRP via a linker (PEG)

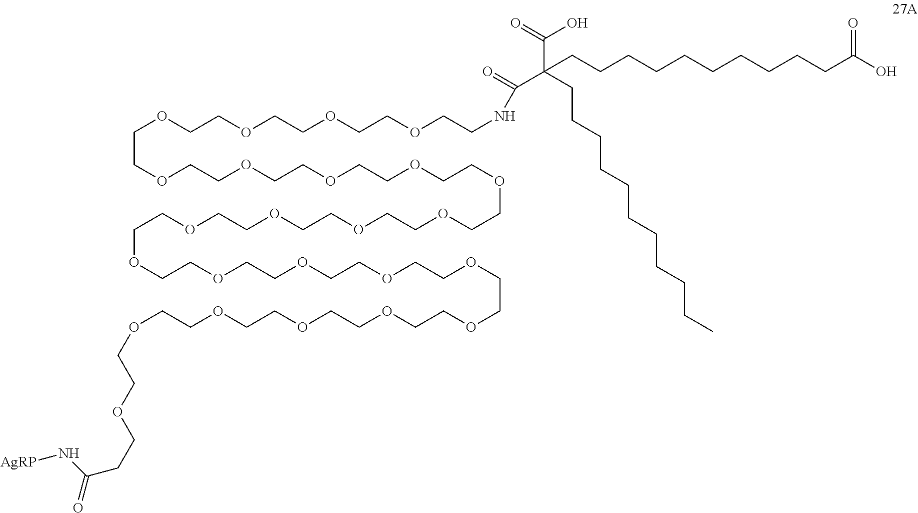

wherein AgRP(83-132) has the following sequence:

Ser-Ser-Arg-Arg-Cys-Val-Arg-Leu-His-Glu-Ser-Cys-Leu-Gly-Gln-Gln-Val-Pro-Cys-Cys-Asp-Pro-Cys-Ala-Thr-Cys-Tyr-Cys-Arg-Phe-Phe-Asn-Ala-Phe-Cys-Tyr-Cys-Arg-Lys-Leu-Gly-Thr-Ala-Met-Asn-Pro-Cys-Ser-Arg-Thr; which contains 5 disulfide bridges at positions C87&C102, C94&C108, C101&C119, C105&C129,C110&C117 Bridges.

Example 5B: di-fatty acid conjugate of AgRP(83-132) (AgRP+2 FA) wherein one fatty acid is attached to the N-terminus of AgRP (i.e. Serine 83) via a linker (PEG) and the other fatty acid is attached to the side chain of Lysine at position 121 via a PEG linker.

To 0.90 ml of a 10 mg/ml solution of AgRP(83-132) (available from R&D Systems™) in pH 4.5 citrate buffer (9 mg, 1.585 μmol) was added 0.80 ml of pH=4.43 acetate buffer followed by a 1.30 ml of a 10 mg/ml solution of 1-37 in H₂O (13 mg, 7.79 μmol). The reaction stirred at room temperature for 16 hours. HRMS (QT2) showed both AgRP+1FA, m/z 7226.3 [M+H] at 1.89 min, and AgRP+2FA, m/z 8778.4 [M+H] at 2.41 min, present. The reaction was filtered through a 4.5 μm frit, combined with a second reaction ran as above (0.881 μmol AgRP, 2.64 μmol 1-37), and purified on preparatory HPLC (Waters Autopure HPLC System; Waters Protein BEH C4 Column, 300 Angstrom, Sum, 10×250 mm; mobile phase: 20-80% ACN in Water, 11 min gradient, 10 mL/min, modified with 0.1% TFA; run time: 15 min; fraction collection: UV 210 nm). Fractions corresponding to AgRP+1FA and AgRP+2FA were isolated, frozen, and lyophilized to give the TFA salts of AgRP+1FA (5A) and AgRP+2FA (5B) as white solids (3.24 mg, 16% AgRP+1FA; 2.26 mg, 9% AgRP+2FA) LCMS-Analytic Method G: (AgRP+1FA) Rt=1.91 mins, MS m/z 7226.4 [M+H]⁺; (AgRP+2FA) Rt=2.43 mins, MS m/z 8778.4 [M+H]⁺.

Labeling Experiment to Determine Position of Attachment of the Fatty Acid.

Labeling at N-terminal Ser residue was confirmed by digesting the reaction mixture with Asp-N (Promega) according to manufacturer protocol. All peptide mapping assays were achieved using a Thermo Dionex Ultimate 3000 LC coupled with a Bruker Maxis Impact Q-TOF mass spectrometer. The separation was performed on an ACQUITY UPLC BEH130 C18 column (2.1×150 mm, 1.7 μm, Waters) kept at 40° C. Flow rate was 0.1 mL/min with 0.1% FA in water as mobile phase A and 0.1% FA in acetonitrile as mobile phase B.

A solution of Asp-N (Promega Part# V162A) was reconstituted in 20 uL of HPLC/MS water (0.1 μg/4). Around 10 μg of sample was diluted to a final volume of 25 μL in 6 M urea, 10 mM dithiothreitol, 5 mM EDTA, and 50 mM Tris_HCl (pH=8.0). After reduction and alkylation, solutions were diluted six times with 50 mM Tris_HCl (pH=8.0), proteolysis was then performed with an additional 1 micrograms of Asp-N. The digests took place overnight at 37 degrees Celsius. LCMS analysis indicated that cleavage had occurred at the N-terminal D positions of wild AgRP and modified AgRP with one addition of fatty acid on each fragment as showed in the following table.

| | | | | Expected | Observed | |
|---|---|---|---|---|---|---|
| peptide sequence | position | mass | RT | m/z | m/z | Charge |
| SSRRCVRLHESCLGQQV | A (1-20) | 2488.13 | 8.1 | 623.04 | 623.03 | 4 |
| PCC | | | | | | |
| DPCATCYCRFFNAFCYCR | A (21-50) | 3783.58 | 10.1 | 757.72 | 757.72 | 5 |
| KLGTAMNPCSRT | | | | | | |
| Modified | | | | | | |
| SSRRCVRLHESCLGQQV | A (1-20) | 4040.12 | 17.4 | 1011.04 | 1011.03 | 4 |
| PCC + fa | | | | | | |
| DPCATCYCRFFNAFCYCR | A (21-50) | 5335.56 | 18.1 | 1068.12 | 1068.12 | 5 |
| KLGTAMNPCSRT + fa | | | | | | |

### Example 6: Activity and Plasma Stability of AgRP Conjugates

The activity and plasma stability of the AgRP conjugates of Example 5A and 5B according to the present invention can be assessed by the following in vitro and in vivo methods described below.

A) HTRF cAMP Assay Protocol:

### Passage of HEK293/MC4R Cells

Cell: HEK293/MC4R stable cell line
Complete medium: DMEM/F12 1:1 (Gibco, Cat. No. 11039, For assay, no-phenol red medium Cat. No. 21041)
   10% FBS (Heat inactivated, Gibco, Cat. No. 10082)
   200 μg/mL Geneticin (Gibco, Cat. No. 10131)
   15 mM Hepes (GIBCO, Cat No. 15630)
   2 mM L-glutamine (GIBCO, Cat No. 25030)
Flask: 150 cm² tissue culture treated flask (Corning, Cat. No. 430825).
   Aspirate conditioned medium
   Wash with 25 mL of DPBS (Gibco, Cat. No. 14190), then aspirate it
      *FBS inhibits Trypsin-EDTA treatment.
   Add 2.5 mL of 0.05% Trypsin-EDTA (Gibco, Cat. No. 25300)
   Leave a few minutes, then tap the flask a few time to detach cells
   Add 25 mL of the complete medium to stop Trypsin-EDTA treatment
      *Cell preparation for assays, no-phenol red complete medium have to be used.
   Pipetting softly a few times to resuspend clumping cells
   Transfer the suspension into a 50 mL centrifuge tube
   Spin down at 1200 rpm for 3 min
   Aspirate supernatant
   Disperse the cells by softly tapping the bottom
   Add 5-10 mL of the complete medium, then resuspend by softly pipetting
      *Cell preparation for assays, no-phenol red complete medium have to be used.
   Transfer 0.5 mL of the suspension into a sample vial for Vi-cell
   Count cell number by using a Vi-cell *Record cell density and viability every time
   Transfer 1-3×10⁶ cells into a new 150 cm flask
      For 3 days: 3×10⁶ cells/flask
      For 4 days: 1×10⁶ cells/flask
   Incubate at 37 C with 5% CO₂
      Cell Seeding for HTRF cAMP Assay (One Day Before Assay)
Prepare cell suspension as in the passage section
Dilute the suspension to 2.34×10⁵ cells/mL
   *13 mL is enough for one 384 well plate.
Dispense 30 μL of the cell suspension into each well of a Poly-D-Lysine BIOCOAT 384-well clear plate (Becton Dickinson, Cat. No. 354660): 7000 cells/well
   *Poly-D-lysine coated plate is essential in this assay.
   *No cell for wells of cAMP standard
Incubate at 37 C with 5% CO₂ over night
   HTRF cAMP Assay

### 1. Preparation of Reagents

1M IBMX

| | |
|---|---|
| IBMX (MW 222.25 g/mol, ACROS Cat. No. 228420010) | 111 mg |
| DMSO (Sigma Aldrich, Cat. No. D2650) | 500 uL |

Store at 4° C.

40 mg/mL BSA solution

| | |
|---|---|
| Bovine serum albumin (Sigma A7030-50G) | 200 mg |
| dH₂O | 5 mL |

Store at 4° C.

1 mg/mL (176 uM) AgRP master solution (in HBSS/2 mg/mL BSA)

| | |
|---|---|
| R&D human AgRP C-terminal (Cat. No. 3726-AG-100) | 100 ug/vial |
| 1x Hanks Buffered Salt Solution (HBSS) | 95 uL |
| (Gibco, Cat. No. 14065, w/Ca and Mg) | |
| 40 mg/mL BSA solution | 5 uL |

Store at 4° C.

2 mM NDP-aMSH master Solution

| | |
|---|---|
| NDP-aMSH (MW 1646.9, Bachem, Cat. No. H1100) | 1 mg/vial |
| dH₂O | 304 uL |

*Once dissolved, dispense 10 uL aliquots into 200 uL tubes, then store at −20° C.

Assay Buffer 1

| | |
|---|---|
| HBSS | 10 mL |
| 1M Hepes (Gibco, Cat. No. 15630) | 0.2 mL |
| 1M IBMX | 20 uL |

*To avoid precipitation of IBMX, please vortex the buffer until fully dissolved.

Assay buffer 2

| | |
|---|---|
| HBSS | 20 mL |
| 1M Hepes (Gibco, Cat. No. 15630) | 0.4 mL |
| 1M IBMX | 40 uL |
| 40 mg/mL BSA solution | 0.25 mL |

*To avoid precipitation of IBMX, please vortex the buffer until fully dissolved.

6 nM NDP-aMSH for IgG titration and AgRP titration

| | |
|---|---|
| 2 uM NDP-aMSH (1000-fold dilution of the master solution) | 10.8 uL |
| Assay buffer 1 | 3600 uL |

*Example for one 384 well assay

120 nM AgRP for IgG titration

| | |
|---|---|
| 10-fold diluted master solution (17.6 uM) | 26 uL |
| Assay buffer2 | 3800 uL |

*Example for one 384-weell plate

NDP-aMSH working solutions for titration (see reagents)
AgRP working solutions for titration (see reagents)
IgG working solutions for titration (see reagents)
cAMP standard solutions (see reagents)

### 2. Assay (2 Step Protocol)

Assay Kit: Cisbio cAMP HiRange HTRF Kit (Cat. No. 62AM6PEB)
Preparation of IgG/AgRP mix (1:1)
   Mix 15 uL of IgG working solutions and 15 uL of 120 nM AgRP, then incubate for 1 hr at ambient temperature
Preparation of assay plate
   Discard culture medium by inverting the 384-well assay plate containing cells on a Wipeall, then tapping in order to remove the culture media.
   Add 100 μL of DPBS to each well and discard in the same manner
      *Once discard PBS, move the next as soon as possible to avoid dry-up
   Transfer 10 μL of the following reagents into each well based on your sample alignment

| | |
|---|---|
| cAMP standard | cAMP standards |
| Negative control for cAMP titration | Diluent in HTRF kit |
| Positive control | cAMP positive control in HTRF kit |
| MSH titration | Assay buffer 2 |
| AgRP titration | AgRP working solutions |
| IgG titaration | IgG/AgRP mixture |
| Negative control for cell assay | Assay buffer 2 |

Flash spindown the 384 well plate at 1200 RPM
Incubated the cells for 15 minutes at an ambient temperature
Add 10 μL of the following reagents into each well based on your sample alignment

| | |
|---|---|
| cAMP standard | Assay buffer 1 |
| Negative control for cAMP titration | Assay buffer 1 |
| Positive control | Assay buffer 1 |
| MSH titration | MSH working solutions |
| AgRP titration | 6 nM MSH solution |
| IgG titaration | 6 nM MSH solution |
| Negative control for cell assay | Assay buffer 1 |

Flash spindown the 384 well plate at 1200 RPM
Incubate the cells for an additional 30 minutes at an ambient temperature
   *This incubation time is not so strict. +/−5 min should be OK according to assay development data.
Add 10 μL of cAMP-d2 (diluted 1:4 in the lysis buffer provided in the kit)
   *Important!! For Negative control, not cAMP-d2, but just the lysis buffer
Add 10 μL of anti-cAMP Cryptate (diluted 1:4 in the lysis buffer provided in the kit)
Flash spindown at 1200 RPM.
Incubate the assay plate for 45-60 min at an ambient temperature.
Transfer 304 of each sample to a tissue culture treated white polystyrene 384-well assay plate (Corning, Cat. No. 3572)
Flash spindown at 1200 RPM.
Measure the fluorescence with a Molecular device M5 or M5e with the following setting.

### Molecular Device M5/M5e Setting

| | |
|---|---|
| Assay type | Time-resolved fluorescence |
| Integ delay | 50 us |
| Integration | 400 us |
| Read | Top read |
| Wave length | Ex 314 nm/Em668 nm Cutoff 630 nm |
| | Ex318 nm/Em570 nm Cutoff 570 nm |
| Auto mix | Off |
| Auto calibration | On |
| Sensitivity | Reading 75 |
| PMT | On |
| Plate | 384 well standard oparque |
| Setting time | off |
| Column wavelength | Column priority |
| priority | |
| Carriage speed | Normal |
| Auto read | Off |

B) MC3 cAMP Assay

### Materials:

Cells: HEK293/MC3R stable cell line
Complete medium: DMEM/F12 1:1 (Gibco, Cat. No. 11039)
10% FBS (Heat inactivated, Gibco, Cat. No. 10082)×
200 μg/mL Geneticin (Gibco, Cat. No. 10131)
2 mM L-glutamine (GIBCO, Cat No. 25030)
   Flask: 150 cm² tissue culture treated flask (Corning, Cat. No. 430825).

### Assay Buffer

| HBSS (Gibco - 14175-095) | 10 mL |
|---|---|
| 1M Hepes (Fisher, Cat. No. BP299-1) | 0.2 mL |
| 500 mM IBMX (MW 222.25 g/mol, ACROS Cat. No. | 40 ul |
| 228420010) | |
| BSA | 0.25% |

### Plates

384 well solid bottom, Greiner bio-one (Cat no.—781080)

### Assay Protocol (Antagonist Protocol):

I. Aspirate conditioned medium
II. Wash with 2.5 mL of DPBS (Gibco, Cat. No. 14190)
III. Add 2 mL of 0.25% Trypsin-EDTA (Gibco, Cat. No. 25200-056)
IV. Leave the flask for few minutes in incubator, tap the flask a few time to detach cells.
V. Add 10 mL of the complete medium to stop Trypsin-EDTA treatment and mix it well by pipetting softly a few times to re-suspend clumping cells
VI. Transfer 1.5 ml of cells into a new 150 cm flask containing 20 ml of complete media
VII. Transfer the remaining suspension into a 50 mL centrifuge tube
VIII. Spin down at 1200 rpm for 4 mins. Aspirate supernatant
IX. Add 6 mL of the assay buffer to the tube and re-suspend the cells by softly pipetting
X. Transfer 0.5 mL of the suspension into a sample vial for Vi-cell and add another 0.5 ml of PBS.
XI. Count cell number by using a Vi-cell *Record cell density and viability every time
   i. Plate cells at 4K/well in 10 ul/well of assay buffer containing IBMX.
   ii. Leave the plate in incubator for ˜30 mins before assay is started on suspension cells.
      Two step cAMP protocol is followed for cAMP determination.

### Procedure

I. To 10 ul/well of cells add 5 ul of AgRP prepared at 3× in assay buffer only to antagonist wells.
II. Add 5 ul of buffer to positive control wells (wells that will have NDP-α-MSH).
III. Incubate the plate at 37° C. for ˜20 mins
IV. Add 5 ul/well of agonist EC80 (NDP-α-MSH) prepared at 4× to wells containing AgRP DRC.
V. Add 5 ul/well of agonist (NDP-α-MSH) DRC prepared at 4× (final highest concentration in plate is 100 nM) for NDP-α-MSH EC50 calculation
VI. Add buffer only to negative control.
VII. Pulse spin the 384 well plate and incubate the cells for 30 minutes in incubator.
VIII. Add 10 μL of the following reagents into each well:
   a. 10 μL of cAMP-d2
   b. *Important!! For Negative control, do not add cAMP-d2, but just the lysis buffer and 10 ul/well of Tb-cryptate
   c. 10 μL of anti-cAMP Cryptate
   d. Pulse spin the plate and incubate for 60 mins at room temperature.

C) In Vivo Assay Description:

10 nanomoles of conjugate was dissolved in 300 L of PBS (Phosphate buffered saline) to form Dosing solution. Dosing solution (300 M) was administered intravenously to male Sprague-Dawley rats via lateral tail vein (corresponding to a dose 10 nanomoles per rat). Blood was collected via tail snip at prescribed times after dosing and immediately placed on wet ice. These samples were centrifuged at 4 C, with supernatant plasma transferred to a fresh tube for analysis.

Bioanalysis:

Standard Curve Preparation:

The two fatty acid conjugates of examples 5A and 5B and one mature human AgRP peptide were used to make standards. Intermediate stock solutions of each AgRP were prepared by diluting the stock labeled peptides in ELISA sample diluent with casein to 100 ug/ml. For assay, intermediates were diluted to a top standard concentration of 2500 pg/mL and then diluted 2-fold serially to 16 points including a zero dose standard in ELISA sample diluent with bovine serum albumin (BSA).

Sample Dilution:

Plasma samples were diluted 10-fold and then 5-fold serially out to 31,250-fold in ELISA sample diluent with BSA.

5B1 Human AgRP ELISA Method:

384 well microplates were coated with anti-human AgRP clone 5B1 overnight at 30 uL/well in 1×PBS at room temperature (RT). Plates were aspirated and blocked with a milk-based blocker at 90 uL/well for 2 hours at RT. All further incubations were carried out at 30 uL/well. Plates were aspirated again and samples and standards were added to the wells for 2 hours at RT. Then the plates were washed three times with a phosphate based wash buffer with tween-20 and a biotinylated goat anti-human AgRP polyclonal antibody was added to the wells to detect the bound AgRP for 2 hours at RT. The plates were washed again and a HRP-labeled streptavidin reagent was added to the wells for 30 minutes at RT. Plates were washed a final time and a chemiluminescent substrate was added to all wells and plates were read immediately on a Spectramx M5 for light output.

Data Analysis:

Raw data was organized and analyzed for basic PK parameters.

### Activity and Stability of AgRP Fatty Acid Conjugates of the Invention According to Assays Described Supra:

TABLE 7

| | | | In vivo Plasma |
|---|---|---|---|
| | MC4R EC50 | MC3R EC50 | stability |
| Peptide | [nM] | [nM] | t½ [h] |
| Example 5A (mono | 18 | 7 | 20 |
| fatty acid | | | |
| conjugate) | | | |
| Example 5B (di | 167 | 65 | 52 |
| fatty acid | | | |
| conjugate) | | | |
| AgRP | 1.7 | 12 | 4.4 |

Unless defined otherwise, the technical and scientific terms used herein have the same meaning as that usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. A method for treating a patient exhibiting one or more wasting disorders, said method comprising administering intranasally to said patient a therapeutically effective amount of an AgRP.

2. The method of claim 1, wherein the metabolic disorder consists of one or more of the following: wasting disorders, anorexia cachexia, anorexia of the aged, anorexia nervosa, cachexia associated with cancer, cachexia associated with AIDS, cachexia associated with heart failure, cachexia associated with cystic fibrosis, cachexia associated with rheumatoid arthritis, cachexia associated with kidney disease, cachexia associated with COPD, cachexia associated with ALS, cachexia associated with renal failure or cachexia associated, or hip fracture.

3. The method of claim 2, wherein the metabolic disorder comprises anorexia cachexia.

4. The method of claim 1 for treating a patient exhibiting one or more wasting disorders, said method comprising administering intranasally to said patient a pharmaceutical composition comprising a therapeutically effective amount of an AgRP, wherein said patient exhibits one or more wasting disorders.

5. The method of claim 4, wherein the metabolic disorder consists of one or more of the following: anorexia cachexia, cachexia associated with cancer, cachexia associated with AIDS, cachexia associated with heart failure, cachexia associated with kidney disease, cachexia associated with COPD, or anorexia nervosa.

6. The method of claim 5, wherein the metabolic disorder comprises anorexia cachexia.

7. A method for achieving one or more of stimulating appetite; decreasing energy expenditure; increasing food intake; increasing body weight; and increasing fat mass in a patient in need, comprising administering intranasally to said patient a therapeutically effective amount of an AgRP.

8. The method of claim 7 for achieving one or more of stimulating appetite; decreasing energy expenditure; increasing food intake; increasing body weight; and increasing fat mass in a patient in need, comprising administering intranasally to said patient a pharmaceutical composition comprising a therapeutically effective amount of an AgRP.

9. A method for treating a patient exhibiting one or more wasting disorders, said method comprising administering intranasally to said patient a therapeutically effective amount of an AgRP in combination one or more of Megestrol acetate; olanzapine; an antibody directed to the myostatin/activinII receptor; Neuropeptide Y, and H3 Relaxin.
